SEQUENCE LISTING (24 sequences)

SEQ ID NO: 1 - DNA, 1580 bp, Lactobacillus sakei
  source 1..1580: mol_type = genomic DNA
cttttaatcgagagtttgatcctggctcaggacgaacgctggcggcgtgcctaatacatgcaagtcgaacgcactctcgtttagattgaaggagcttgctcctgattgataaacatttgagtgagtggcggacgggtgagtaacacgtgggtaacctgccctaaagtgggggataacatttggaaacagatgctaataccgcataaaacctaacaccgcatggtgtagggttgaaagatggtttcggctatcactttaggatggacccgcggtgcattagttagttggtgaggtaaaggctcaccaagaccgtgatgcatagccgacctgagagggtaatcggccacactgggactgagacacggcccagactcctacgggaggcagcagtagggaatcttccacaatggacgaaagtctgatggagcaacgccgcgtgagtgaagaaggttttcggatcgtaaaactctgttgttggagaagaatgtatctgatagtaactgatcaggtagtgacggtatccaaccagaaagccacggctaactacgtgccagcagccgcggtaatacgtaggtggcaagcgttgtccggatttattgggcgtaaagcgagcgcaggcggtttcttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagcatgggtagcaaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattactagttgccagcatttagttgggcactctagtgagactgccggtgacaaaccggaggaaggtggggacgacgtcaaatcatcatgccccttatgacctgggctacacacgtgctacaatggatggtacaacgagttgcgagaccgcgaggtttagctaatctcttaaaaccattctcagttcggattgtaggctgcaactcgcctacatgaagccggaatcgctagtaatcgcggatcagcatgccgcggtgaatacgttcccgggccttgtacacaccgcccgtcacaccatgagagtttgtaacacccaaagccggtgaggtaacccttcggggagccagccgtctaaggtgggacagatgattagggtgaagtcgtaacaaggtagccgtaggagaacctgcggctggatcacctcctttcta

SEQ ID NO: 2 - DNA, 23 bp, synthetic construct
  source 1..23: mol_type = other DNA
ggatgatacaggacctgacaaag

SEQ ID NO: 3 - DNA, 19 bp, synthetic construct
  source 1..19: mol_type = other DNA
ccgaggcgttggtgatgat

SEQ ID NO: 4 - DNA, 20 bp, synthetic construct
  source 1..20: mol_type = other DNA
actcgctggaacacagatgc

SEQ ID NO: 5 - DNA, 21 bp, synthetic construct
  source 1..21: mol_type = other DNA
tctgagagcctgagacctttt

SEQ ID NO: 6 - DNA, 19 bp, synthetic construct
  source 1..19: mol_type = other DNA
cggacaaacggctcactct

SEQ ID NO: 7 - DNA, 21 bp, synthetic construct
  source 1..21: mol_type = other DNA
ggacccgcatgaatcgactat

SEQ ID NO: 8 - DNA, 23 bp, synthetic construct
  source 1..23: mol_type = other DNA
acactcactcagatcaaagatgc

SEQ ID NO: 9 - DNA, 23 bp, synthetic construct
  source 1..23: mol_type = other DNA
ccctttacactgggcttatgttt

SEQ ID NO: 10 - DNA, 23 bp, synthetic construct
  source 1..23: mol_type = other DNA
tgtttgaagagacccagattgga

SEQ ID NO: 11 - DNA, 20 bp, synthetic construct
  source 1..20: mol_type = other DNA
atggccctcctgacgatcat

SEQ ID NO: 12 - DNA, 1149 bp, Lactobacillus sakei
  source 1..1149: mol_type = genomic DNA
cccctgtgttccactatagacggctggctccccgaagggttacctcaccggctttgggtgttacaaactctcatggtgtgacgggcggtgtgtacaaggcccgggaacgtattcaccgcggcatgctgatccgcgattactagcgattccggcttcatgtaggcgagttgcagcctacaatccgaactgagaatggttttaagagattagctaaacctcgcggtctcgcaactcgttgtaccatccattgtagcacgtgtgtagcccaggtcataaggggcatgatgatttgacgtcgtccccaccttcctccggtttgtcaccggcagtctcactagagtgcccaactaaatgctggcaactagtaataagggttgcgctcgttgcgggacttaacccaacatctcacgacacgagctgacgacaaccatgcaccacctgtcactttgtccccgaagggaaagctctatctctagagtggtcaaaggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcggcactgaagggcggaaaccctccaacacctagcactcatcgtttacggcatggactaccagggtatctaatcctgtttgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaggctttcacatcagacttaagaaaccgcctgcgctcgctttacgcccaataaatccggacacgctgccacctacgtattaccgcggctgctgcacgtagtagcgtgctttctggtgatacgtcactactgatcagtactatcagatacattcttctccacaacagaagtttacgatcgaaacctttcttcactccacgcggcggtgctccatccgacttctgtctatggggagatcatcgatctcctccgcgcgcggtcagagaaaaaaaaaaag

SEQ ID NO: 13 - DNA, 1078 bp, Lactobacillus sakei
  source 1..1078: mol_type = genomic DNA
cccaggctgcttcagctatgatcagctggctccccgaagggttacctcaccggctttgggtgttacaaactctcatggtgtgacgggcggtgtgtacaaggcccgggaacgtattcaccgcggcatgctgatccgcgattactagcgattccggcttcatgtaggcgagttgcagcctacaatccgaactgagaatggttttaagagattagctaaacctcgcggtctcgcaactcgttgtaccatccattgtagcacgtgtgtagcccaggtcataaggggcatgatgatttgacgtcgtccccaccttcctccggtttgtcaccggcagtctcactagagtgcccaactaaatgctggcaactagtaataagggttgcgctcgttgcgggacttaacccaacatctcacgacacgagctgacgacaaccatgcaccacctgtcactttgtccccgaagggaaagctctatctctagagtggtcaaaggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcggcactgaagggcggaaaccctccaacacctagcactcatcgtttacggcatggactaccagggtatctaatcctgtttgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaggctttcacatcagacttaagaaaccgcctgcgctcgctttacgcccaataaatccagacaacgcttgccacctacgtataccgcggctgctggcacgtagttagccgtggctttctggttggataccgtcactacctgatcagttactatcagatacattcttctccaccacagaagttttacgatccgaaaaccttcctc

SEQ ID NO: 14 - DNA, 1081 bp, Lactobacillus sakei
  source 1..1081: mol_type = genomic DNA
cccatcctgtcagctatgaccagctggctcccgaagggttacctcaccggctttgggtgttacaaactctcatggtgtgacgggcggtgtgtacaaggcccgggaacgtattcaccgcggcatgctgatccgcgattactagcgattccggcttcatgtaggcgagttgcagcctacaatccgaactgagaatggttttaagagattagctaaacctcgcggtctcgcaactcgttgtaccatccattgtagcacgtgtgtagcccaggtcataaggggcatgatgatttgacgtcgtccccaccttcctccggtttgtcaccggcagtctcactagagtgcccaactaaatgctggcaactagtaataagggttgcgctcgttgcgggacttaacccaacatctcacgacacgagctgacgacaaccatgcaccacctgtcactttgtccccgaagggaaagctctatctctagagtggtcaaaggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcggcactgaagggcggaaaccctccaacacctagcactcatcgtttacggcatggactaccagggtatctaatcctgtttgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcacttcttcggttgagccgaaggctttcacatcagacttaagaaaccgcctgcgctcgctttacgcccaataaatccggacaacgcttgccacctacgtattaccgcggctgctggcacgtagttagccgtgctttctggttggataccgtcactacctgatcagttactatcagatacattcttctccaacaacagagttttacgatccggaaaaccttcttcattca

SEQ ID NO: 15 - DNA, 1154 bp, Bacillus coagulans
  source 1..1154: mol_type = genomic DNA
caacacctgtcacctatcagcaagctggctccgtaaggttacctcaccgacttcgggtgttacaaactctcgtggtgtgacgggcggtgtgtacaaggcccgggaacgtattcaccgcggcatgctgatccgcgattactagcgattccggcttcatgcaggcgggttgcagcctgcaatccgaactgggaatggttttctgggattggcttaacctcgcggtctcgcagccctttgtaccatccattgtagcacgtgtgtagcccaggtcataaggggcatgatgatttgacgtcatccccaccttcctccggtttgtcaccggcagtcaccttagagtgcccaactgaatgctggcaactaaggtcaagggttgcgctcgttgcgggacttaacccaacatctcacgacacgagctgacgacaaccatgcaccacctgtcactctgtcccccgaaggggaaggccctgtctccagggaggtcagaggatgtcaagacctggtaaggttcttcgcgttgcttcgaattaaaccacatgctccaccgcttgtgcgggcccccgtcaattcctttgagtttcagccttgcggccgtactccccaggcggagtgcttaatgcgttagctgcagcactaaagggcggaaaccctctaacacttagcactcatcgtttacggcgtggactaccagggtatctaatcctgtttgctccccacgctttcgcgcctcagcgtcagttacagaccagagagccgccttcgccactggtgttcctccacatctctacgcatttcaccgctacacgtggaattccactctcctcttctgcactcaagcctcccagtttccaatgaccgcttgcggttgagccgcaagatttcacatcagacttaagaagccgcctgcgcgcgctttacgcccaataattcccggacacgcttgccacctacgtattaccgcggctgctggcacgtagttagccgtggctttctggccgggtaccgtcaggcaccgccctgtcgacggcactgtctccccgggcacagagttttacgacccgagcttcttcactccacgcgggcgtgctccgtcagactttcgtcatgcggaggatcctactgtctccctcgcgcgagtcatgatgaaaatcaaa

SEQ ID NO: 16 - DNA, 1153 bp, Bacillus coagulans
  source 1..1153: mol_type = genomic DNA
ccatgcttgttccaccgtatcgagcggctggctccgtaaggttaactcaccgacttcgggtgttacaaactctcgtggtgtgacgggcggtgtgtacaaggcccgggaacgtattcaccgcggcatgctgatccgcgattactagcgattccggcttcatgcaggcgggttgcagcctgcaatccgaactgggaatggttttctgggattggcttaacctcgcggtctcgcagccctttgtaccatccattgtagcacgtgtgtagcccaggtcataaggggcatgatgatttgacgtcatccccaccttcctccggtttgtcaccggcagtcaccttagagtgcccaactgaatgctggcaactaaggtcaagggttgcgctcgttgcgggacttaacccaacatctcacgacacgagctgacgacaaccatgcaccacctgtcactctgtcccccgaaggggaaggccctgtctccagggaggtcagaggatgtcaagacctggtaaggttcttcgcgttgcttcgaattaaaccacatgctccaccgcttgtgcgggcccccgtcaattcctttgagtttcagccttgcggccgtactccccaggcggagtgcttaatgcgttagctgcagcactaaagggcggaaaccctctaacacttagcactcatcgtttacggcgtggactaccagggtatctaatcctgtttgctccccacgctttcgcgcctcagcgtcagttacagaccagagagccgccttcgccactggtgttcctccacatctctacgcatttcaccgctacacgtggaattccactctcctcttctgcactcaagcctcccagtttccaatgaccgcttgcggttgagccgcaagatttcacatcagacttaagaagccgcctgcgcgcgctttacgcccaatattccggacacgcttgccacctacgtattaccgcggctgctggcacgtagttagccgtggctttctggccgggtacgtcaagcaccgccctgttcgacggcactgtctcccggcaacagagttttacgacccgagctcctcactcacgcggcgtggcttcggtccgacctccgttcattgcgagatctactgtgtctcccctgaggagagtcagaacacaaatag

SEQ ID NO: 17 - DNA, 1013 bp, Bacillus coagulans
  source 1..1013: mol_type = genomic DNA
cacgtttggctgcacctatcagcggctggctccgtaaggttacctcaccgacttcgggtgttacaaactctcgtggtgtgacgggcggtgtgtacaaggcccgggaacgtattcaccgcggcatgctgatccgcgattactagcgattccggcttcatgcaggcgggttgcagcctgcaatccgaactgggaatggttttctgggattggcttaacctcgcggtctcgcagccctttgtaccatccattgtagcacgtgtgtagcccaggtcataaggggcatgatgatttgacgtcatccccaccttcctccggtttgtcaccggcagtcaccttagagtgcccaactgaatgctggcaactaaggtcaagggttgcgctcgttgcgggacttaacccaacatctcacgacacgagctgacgacaaccatgcaccacctgtcactctgtcccccgaaggggaaggccctgtctccagggaggtcagaggatgtcaagacctggtaaggttcttcgcgttgcttcgaattaaaccacatgctccaccgcttgtgcgggcccccgtcaattcctttgagtttcagccttgcggccgtactccccaggcggagtgcttaatgcgttagctgcagcactaaagggcggaaaccctctaacacttagcactcatcgtttacggcgtggactaccagggtatctaatcctgtttgctccccacgctttcgcgcctcagcgtcagttacagaccagagagccgccttcgccactggtgttcctccacatctctacgcatttcaccgctacacgtggaattccactctcctcttctgcactcaagcctcccagtttccaatgaccgcttgcggttgagccgcaagatttcacatcagacttaagaagccgcctgcgcgcgctttacgcccaataattccggacacgcttgccacctacgtattaccgcggctgctggcacgtagttagccgtggctttctgggccgggtaccgtcaagca

SEQ ID NO: 18 - DNA, 972 bp, Bacillus coagulans
  source 1..972: mol_type = genomic DNA
caacattttgtctggaagctatcagcggctggctccgtaagagttacctcaccgacttcgggtgttacaaactctcgtggtgtgacgggcggtgtgtacaaggcccgggaacgtattcaccgcggcatgctgatccgcgattactagcgattccggcttcatgcaggcgggttgcagcctgcaatccgaactgggaatggttttctgggattggcttaacctcgcggtctcgcagccctttgtaccatccattgtagcacgtgtgtagcccaggtcataaggggcatgatgatttgacgtcatccccaccttcctccggtttgtcaccggcagtcaccttagagtgcccaactgaatgctggcaactaaggtcaagggttgcgctcgttgcgggacttaacccaacatctcacgacacgagctgacgacaaccatgcaccacctgtcactctgtcccccgaaggggaaggccctgtctccagggaggtcagaggatgtcaagacctggtaaggttcttcgcgttgcttcgaattaaaccacatgctccaccgcttgtgcgggcccccgtcaattcctttgagtttcagccttgcggccgtactccccaggcggagtgcttaatgcgttagctgcagcactaaagggcggaaaccctctaacacttagcactcatcgtttacggcgtggactaccagggtatctaatcctgtttgctccccacgctttcgcgcctcagcgtcagttacagaccagagagccgccttcgccactggtgttcctccacatctctacgcatttcaccgctacacgtggaattccactctcctcttctgcactcaagcctcccagtttccaatgaccgcttgcggttgagccgcaagatttcaacatcagacttaagaagccgcctgcgcgcgctttacgcccaataattccggacaacgcttgccacctacgtattaccgcggctg

SEQ ID NO: 19 - DNA, 1067 bp, Bacillus coagulans
  source 1..1067: mol_type = genomic DNA
cagccattgtgtaggtatcagcggctggctccgtaagagtaacctcaccgacttcgggtgttacaaactctcgtggtgtgacgggcggtgtgtacaaggcccgggaacgtattcaccgcggcatgctgatccgcgattactagcgattccggcttcatgcaggcgggttgcagcctgcaatccgaactgggaatggttttctgggattggcttaacctcgcggtctcgcagccctttgtaccatccattgtagcacgtgtgtagcccaggtcataaggggcatgatgatttgacgtcatccccaccttcctccggtttgtcaccggcagtcaccttagagtgcccaactgaatgctggcaactaaggtcaagggttgcgctcgttgcgggacttaacccaacatctcacgacacgagctgacgacaaccatgcaccacctgtcactctgtcccccgaaggggaaggccctgtctccagggaggtcagaggatgtcaagacctggtaaggttcttcgcgttgcttcgaattaaaccacatgctccaccgcttgtgcgggcccccgtcaattcctttgagtttcagccttgcggccgtactccccaggcggagtgcttaatgcgttagctgcagcactaaagggcggaaaccctctaacacttagcactcatcgtttacggcgtggactaccagggtatctaatcctgtttgctccccacgctttcgcgcctcagcgtcagttacagaccagagagccgccttcgccactggtgttcctccacatctctacgcatttcaccgctacacgtggaaattccactctcctcttctgcactcaagcctcccagtttccaatgaccgcttgcggttgagccgcaagatttcacatcagacttaagaagccgcctgcgcgcgctttacgcccaataattccggacacgcttgccacctacgtattaccgcgctgctggcacgtagttagccgtgctttctgccgggtaccgtcagcaccgccctgttcgaacggcacttgttcttccccggcaacagaagtttttacgacccgaa

SEQ ID NO: 20 - DNA, 1047 bp, Lactobacillus casei
  source 1..1047: mol_type = genomic DNA
caacgtttggtcaggttagacggctcgctccctaaaagggttacgccaccggcttcgggtgttacaaactctcatggtgtgacgggcggtgtgtacaaggcccgggaacgtattcaccgcggcgtgctgatccgcgattactagcgattccgacttcgtgtaggcgagttgcagcctacagtccgaactgagaatggctttaagagattagcttgacctcgcggtctcgcaactcgttgtaccatccattgtagcacgtgtgtagcccaggtcataaggggcatgatgatttgacgtcatccccaccttcctccggtttgtcaccggcagtcttactagagtgcccaactaaatgctggcaactagtcataagggttgcgctcgttgcgggacttaacccaacatctcacgacacgagctgacgacaaccatgcaccacctgtcattttgcccccgaaggggaaacctgatctctcaggtgatcaaaagatgtcaagacctggtaaggttcttcgcgttgcttcgaattaaaccacatgctccaccgcttgtgcgggcccccgtcaattcctttgagtttcaaccttgcggtcgtactccccaggcggaatgcttaatgcgttagctgcggcactgaagggcggaaaccctccaacacctagcattcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcgcttcctcggttaagccgagggctttcacatcagacttaaaaaaccgcctgcgctcgctttacgcccaataaatccggataacgcttgccacctacgtattaccgcggctgctggcacgtagttagccgtggctttctggttggataccgtcacgccgacacagttactctgccgaccattcttctccacca

SEQ ID NO: 21 - DNA, 1079 bp, Lactobacillus casei
  source 1..1079: mol_type = genomic DNA
caaagttgtcagcaatagacggctcgctccctaaaagggttacgccaccggcttcgggtgttacaaactctcatggtgtgacgggcggtgtgtacaaggcccgggaacgtattcaccgcggcgtgctgatccgcgattactagcgattccgacttcgtgtaggcgagttgcagcctacagtccgaactgagaatggctttaagagattagcttgacctcgcggtctcgcaactcgttgtaccatccattgtagcacgtgtgtagcccaggtcataaggggcatgatgatttgacgtcatccccaccttcctccggtttgtcaccggcagtcttactagagtgcccaactaaatgctggcaactagtcataagggttgcgctcgttgcgggacttaacccaacatctcacgacacgagctgacgacaaccatgcaccacctgtcattttgcccccgaaggggaaacctgatctctcaggtgatcaaaagatgtcaagacctggtaaggttcttcgcgttgcttcgaattaaaccacatgctccaccgcttgtgcgggcccccgtcaattcctttgagtttcaaccttgcggtcgtactccccaggcggaatgcttaatgcgttagctgcggcactgaagggcggaaaccctccaacacctagcattcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcgcttcctcggttaagccgagggctttcacatcagacttaaaaaaccgcctgcgctcgctttacgcccaataaatcccggataacgcttgccacctacgtattaccgcggctgctggcacgtagttagccgtggctttctggttggataccgtcacgccgacacagttactctgccgaccattcttctccaacaacagaagttttacgacccggaaagccctcctc

SEQ ID NO: 22 - DNA, 1079 bp, Lactobacillus casei
  source 1..1079: mol_type = genomic DNA
ggcgataccttgttgtacctgtccgacgagctcgctccctaaaagggttacgccaccggcttcgggtgttacaaactctcatggtgtgacgggcggtgtgtacaaggcccgggaacgtattcaccgcggcgtgctgatccgcgattactagcgattccgacttcgtgtaggcgagttgcagcctacagtccgaactgagaatggctttaagagattagcttgacctcgcggtctcgcaactcgttgtaccatccattgtagcacgtgtgtagcccaggtcataaggggcatgatgatttgacgtcatccccaccttcctccggtttgtcaccggcagtcttactagagtgcccaactaaatgctggcaactagtcataagggttgcgctcgttgcgggacttaacccaacatctcacgacacgagctgacgacaaccatgcaccacctgtcattttgcccccgaaggggaaacctgatctctcaggtgatcaaaagatgtcaagacctggtaaggttcttcgcgttgcttcgaattaaaccacatgctccaccgcttgtgcgggcccccgtcaattcctttgagtttcaaccttgcggtcgtactccccaggcggaatgcttaatgcgttagctgcggcactgaagggcggaaaccctccaacacctagcattcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcgcttcctcggttaagccgagggctttcacatcagacttaaaaaaccgcctgcgctcgctttacgcccaataaatccggataacgcttgccacctacgtattaccgcggctgctggcacgtagttagccgtggctttctggttggataccgtcacgccgacacagttactctgcgaccattcttctcacacagagttttacgacccgaaagccttctt

SEQ ID NO: 23 - DNA, 1093 bp, Lactobacillus casei
  source 1..1093: mol_type = genomic DNA
cccccgcatgctgcggcagcgttcgacaagctcgctccctaaaagggttacgccaccggcttcgggtgttacaaactctcatggtgtgacgggcggtgtgtacaaggcccgggaacgtattcaccgcggcgtgctgatccgcgattactagcgattccgacttcgtgtaggcgagttgcagcctacagtccgaactgagaatggctttaagagattagcttgacctcgcggtctcgcaactcgttgtaccatccattgtagcacgtgtgtagcccaggtcataaggggcatgatgatttgacgtcatccccaccttcctccggtttgtcaccggcagtcttactagagtgcccaactaaatgctggcaactagtcataagggttgcgctcgttgcgggacttaacccaacatctcacgacacgagctgacgacaaccatgcaccacctgtcattttgcccccgaaggggaaacctgatctctcaggtgatcaaaagatgtcaagacctggtaaggttcttcgcgttgcttcgaattaaaccacatgctccaccgcttgtgcgggcccccgtcaattcctttgagtttcaaccttgcggtcgtactccccaggcggaatgcttaatgcgttagctgcggcactgaagggcggaaaccctccaacacctagcattcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcgcttcctcggttaagccgagggctttcacatcagacttaaaaaaccgcctgcgctcgctttacgcccaataaatccggataacgcttgccacctacgtattaccgcggctgctggcacgtagttagccgtggctttctggttggataccgtcacgccgacacagttactctgccgaccatctctcaacaacagagttttacgacccgaaagcttcttcactcacgcggcgt

SEQ ID NO: 24 - DNA, 1145 bp, Lactobacillus casei
  source 1..1145: mol_type = genomic DNA
caaccttgtcaccttagacggctcgctccctaaaagggttacgccaccggcttcgggtgttacaaactctcatggtgtgacgggcggtgtgtacaaggcccgggaacgtattcaccgcggcgtgctgatccgcgattactagcgattccgacttcgtgtaggcgagttgcagcctacagtccgaactgagaatggctttaagagattagcttgacctcgcggtctcgcaactcgttgtaccatccattgtagcacgtgtgtagcccaggtcataaggggcatgatgatttgacgtcatccccaccttcctccggtttgtcaccggcagtcttactagagtgcccaactaaatgctggcaactagtcataagggttgcgctcgttgcgggacttaacccaacatctcacgacacgagctgacgacaaccatgcaccacctgtcattttgcccccgaaggggaaacctgatctctcaggtgatcaaaagatgtcaagacctggtaaggttcttcgcgttgcttcgaattaaaccacatgctccaccgcttgtgcgggcccccgtcaattcctttgagtttcaaccttgcggtcgtactccccaggcggaatgcttaatgcgttagctgcggcactgaagggcggaaaccctccaacacctagcattcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactgtcctcttctgcactcaagtttcccagtttccgatgcgcttcctcggttaagccgagggctttcacatcagacttaaaaaaccgcctgcgctcgctttacgcccaataaatccggataacgcttgccacctacgtattaccgcggctgctggcacgtagttagccgtggctttctggttggataccgtcacgccgacacagttactctgccgaccattcttctccaacaacagagtttacgacccgaaagccttcttcactcacgcggcgtgctccatcagacttgcgtccattgtggagatctatgcccccgggggtataaaaaaaga

(19) United States
(12) Patent Application Publication
(10) Pub. No.: US 2023/0218504 A1
(43) Pub. Date: Jul. 13, 2023

(54) LACTOBACILLUS SAKEI AND METHOD FOR SPERMIDINE PRODUCTION WITH LACTOBACILLUS SAKEI OR METABOLITE THEREOF

(71) Applicant: TCI CO., LTD., Taipei (TW)

(72) Inventors: YUNG-HSIANG LIN, Taipei (TW); DI CHANG, Taipei (TW)

(21) Appl. No.: 18/147,722

(22) Filed: Dec. 29, 2022

Related U.S. Application Data
(60) Provisional application No. 63/298,222, filed on Jan. 11, 2022.

(51) Int. Cl. A61K 8/99; C12N 1/20; A61Q 19/08; A61Q 19/00

(52) U.S. Cl. CPC A61K 8/99; C12N 1/205; A61Q 19/08; A61Q 19/007; C12R 2001/225

(57) ABSTRACT

Lactobacillus sakei is provided, wherein the Lactobacillus sakei is Lactobacillus sakei TCI147 with an accession number of DSM 33913. A method for spermidine production with Lactobacillus sakei or metabolites thereof is provided, wherein the Lactobacillus sakei is Lactobacillus sakei TCI147 with an accession number of DSM 33913.

### CROSS-REFERENCE TO RELATED APPLICATION

This application claims the benefit of U.S. provisional application Ser. No. 63/298,222, filed on Jan. 11, 2022. The entirety of the above-mentioned patent application is hereby incorporated by reference herein and made a part of the specification.

### REFERENCE OF AN ELECTRONIC SEQUENCE LISTING

The contents of the electronic sequence listing (P222871USI_ST26.xml; Size: 37 KB; and Date of Creation: Dec. 29, 2022) is herein incorporated by reference in its entirety.

### BACKGROUND

### Technical Field

The present disclosure relates to Lactobacillus sakei, and in particular to Lactobacillus sakei and a method for spermidine production with the Lactobacillus sakei or a metabolite thereof.

### Related Art

Autophagy is an anti-aging mechanism of cells. The cells obtain enough nutrients, by breaking down their own unnecessary and dysfunctional structures and organelles, to survive damage of stress. However, with age, the autophagy efficiency of old cells decreases, leading to cell damage and accumulation of senescent cells.

Spermidine is a polyamine crystalline compound, which can effectively induce the autophagy, and has been proved to be a potential anti-aging substance. The ability of a human body to produce the spermidine gradually decreases with age, and therefore, we need to be supplemented with exogenous spermidine. However, the spermidine ingested in a general diet has been digested and hydrolyzed by the digestive tract, gastric acid and trypsin, and there is not much spermidine left for intestinal absorption.

In order to solve the above problems, those skilled in the art urgently need to develop a scientifically based and effective probiotic product to benefit the vast number of people in need.

### SUMMARY

In view of this, the present disclosure provides Lactobacillus sakei, and a method for spermidine production with the Lactobacillus sakei or a metabolite thereof.

In some embodiments, Lactobacillus sakei is provided, wherein the Lactobacillus sakei is Lactobacillus sakei TCI147 with an accession number of DSM 33913.

In some embodiments, use of Lactobacillus sakei or a metabolite thereof to prepare a composition for spermidine production is provided, wherein the Lactobacillus sakei is Lactobacillus sakei TCI147 with an accession number of DSM 33913.

In some embodiments, a method for spermidine production includes administering to a subject in need thereof a composition including the Lactobacillus sakei or the metabolite thereof. The Lactobacillus sakei is the Lactobacillus sakei TCI147 with the accession number of DSM 33913.

In some embodiments, the Lactobacillus sakei is used for delaying cellular aging or oxidation.

In some embodiments, the Lactobacillus sakei is used for enhancing mitochondrial activity.

In some embodiments, the Lactobacillus sakei is used for promoting autophagy.

In some embodiments, the Lactobacillus sakei is used for preventing and/or reducing oxidative stress of cells.

In some embodiments, the Lactobacillus sakei is used for increasing glutathione (GSH) content.

In some embodiments, the Lactobacillus sakei is used for enhancing glutathione S-transferase (GST) activity.

In some embodiments, the Lactobacillus sakei is used for reducing malondialdehyde (MDA) content and/or increasing glutathione S-transferase in red blood cells (GST-RBC) content.

In some embodiments, the Lactobacillus sakei is used for delaying skin aging.

In some embodiments, the Lactobacillus sakei is used for increasing skin collagen content.

In some embodiments, the Lactobacillus sakei is used for fading skin spots.

In some embodiments, the Lactobacillus sakei is used for reducing skin roughness.

In some embodiments, the Lactobacillus sakei is used for reducing skin fine lines and/or wrinkles.

In some embodiments, the Lactobacillus sakei is used for promoting skin moisture retention capacity.

In some embodiments, an effective dose of the Lactobacillus sakei is 100 mg/day.

In summary, the Lactobacillus sakei in any embodiment can produce spermidine. In some embodiments, the Lactobacillus sakei or metabolites thereof in any embodiment is suitable for preparing a composition for spermidine production. In some embodiments, a method for spermidine production includes administering to a subject in need thereof a composition including the Lactobacillus sakei or the metabolites thereof in any embodiment. In other words, the aforementioned composition has a spermidine production function. In some embodiments, the Lactobacillus sakei, the metabolites thereof or the composition prepared therefrom further has one or more of the following functions: delaying cellular aging or oxidation, enhancing mitochondrial activity, promoting autophagy, preventing and/or reducing oxidative stress of the cells, increasing GSH content, enhancing GST activity, reducing MDA content, increasing GST-RBC content, delaying skin aging, increasing skin collagen content, fading skin spots, reducing skin roughness, reducing skin fine lines and/or wrinkles and promoting skin moisture retention capacity. In some embodiments, a method for delaying cellular aging or oxidation, enhancing mitochondrial activity, promoting autophagy, preventing and/or delaying oxidative stress of the cells, increasing GSH content, enhancing GST activity, reducing MDA content, increasing GST-RBC content, delaying skin aging, increasing skin collagen content, fading skin spots, reducing skin roughness, reducing skin fine lines and/or wrinkles and promoting skin moisture retention capacity includes administering to a subject in need thereof a composition including the Lactobacillus sakei or the metabolites thereof.

### BRIEF DESCRIPTION OF THE DRAWINGS

FIG. 1 is a bar chart showing the cell experimental results of relative NADSYN1 gene expression.

FIG. 2 is a bar chart showing the cell experimental results of relative ATG8 gene expression.

FIG. 3 is a bar chart showing the cell experimental results of relative FOXO gene expression.

FIG. 4 is a bar chart showing the cell experimental results of relative CCT6A gene expression.

FIG. 5 is a bar chart showing the cell experimental results of relative CCT7 gene expression.

FIG. 6 is a bar chart showing the cell experimental results of relative glutathione S-transferase activity.

FIG. 7 is a bar chart showing the cell experimental results of relative glutathione content.

FIG. 8 is a bar chart showing the cell experimental results of relative retinal epithelial cell size.

FIG. 9 is a bar chart showing the cell experimental results of relative hair follicle cell size.

FIG. 10 is a fluorescent staining photo showing the cell experimental results of sizes of retinal epithelial cells (A) and hair follicle cells (B).

FIG. 11 is a bar chart showing the cell experimental results of relative collagen secretion content.

FIG. 12 is a fluorescent staining photo showing the cell experimental results of collagen content.

FIG. 13 is a bar chart showing the human experimental results of malondialdehyde content at weeks 0, 4, and 8.

FIG. 14 is a bar chart showing the human experimental results of glutathione S-transferase in red blood cells content at weeks 0, 4, and 8.

FIG. 15 is a bar chart showing the human experimental results of relative skin brown spot level at weeks 0, 4, and 8.

FIG. 16 is a bar chart showing the human experimental results of relative skin texture level at weeks 0, 4, and 8.

FIG. 17 is a bar chart showing the human experimental results of relative skin fine line level at weeks 0, 4, and 8.

FIG. 18 is a photo showing the human experimental results of fine lines around the eyes at weeks 0 and 8.

FIG. 19 is a bar chart showing the human experimental results of relative skin collagen density at weeks 0 and 8.

FIG. 20 is a bar chart showing the human experimental results of relative skin hydration at weeks 0 and 8.

FIG. 21 is a bar chart showing the cell experimental results of spermidine production.

### DETAILED DESCRIPTION

Some of specific implementations of the present disclosure will be described below. Without departing from the spirit of the present disclosure, the present disclosure still can be practiced in many different forms of aspects, and the scope of protection should not be limited to the conditions specified in the specification.

In some embodiments, Lactobacillus sakei is a dominant lactic acid bacteria strain in a sake fermentation process, which makes high-quality sake by producing lactic acid and flavor substances. Moreover, the sake brewed from rice is rich in many amino acids.

In some embodiments, Lactobacillus sakei is provided, which is Lactobacillus sakei TCI147. The Lactobacillus sakei TCI147 was deposited in the Food Industry Research and Development Institute (Taiwan) under an accession number of BCRC 911063, and in the Deutsche Sammlung von Mikroorganismen and Zellkulturen (DSMZ) under an accession number of DSM 33913.

In some embodiments, the aforementioned Lactobacillus sakei TCI147 is isolated from Lentinus edodes.

In some embodiments, the aforementioned Lactobacillus sakei or the metabolite thereof has a spermidine production effect. In other words, the Lactobacillus sakei or the metabolite thereof is suitable for preparing a composition for spermidine production.

In some embodiments, a method for spermidine production includes administering to a subject in need thereof a composition including the Lactobacillus sakei or the metabolite thereof.

In some embodiments, the aforementioned Lactobacillus sakei is used for delaying cellular aging or oxidation.

In some embodiments, the aforementioned Lactobacillus sakei is used for enhancing mitochondrial activity.

In some embodiments, the aforementioned Lactobacillus sakei is used for promoting autophagy.

In some embodiments, the aforementioned Lactobacillus sakei is used for preventing and/or delaying oxidative stress of cells.

In some embodiments, the aforementioned Lactobacillus sakei is used for increasing glutathione (GSH) content.

In some embodiments, the aforementioned Lactobacillus sakei is used for enhancing glutathione S-transferase (GST) activity.

In some embodiments, the aforementioned Lactobacillus sakei is used for reducing malondialdehyde (MDA) content and/or increasing glutathione S-transferase in red blood cells (GST-RBC) content.

In some embodiments, the aforementioned Lactobacillus sakei is used for delaying skin aging.

In some embodiments, the aforementioned Lactobacillus sakei is used for increasing skin collagen content.

In some embodiments, the aforementioned Lactobacillus sakei is used for fading skin spots.

In some embodiments, the aforementioned Lactobacillus sakei is used for reducing skin roughness.

In some embodiments, the aforementioned Lactobacillus sakei is used for reducing skin fine lines and/or wrinkles.

In some embodiments, the aforementioned Lactobacillus sakei is used for promoting skin moisture retention capacity.

In some embodiments, the aforementioned Lactobacillus sakei or the metabolite thereof further has abilities of delaying cellular aging or oxidation, enhancing mitochondrial activity, promoting autophagy, preventing and/or reducing oxidative stress of the cells, increasing GSH content, enhancing GST activity, reducing MDA content, increasing GST-RBC content, delaying skin aging, increasing skin collagen content, fading skin spots, reducing skin roughness, reducing skin fine lines and/or wrinkles, promoting skin moisture retention capacity or any combination thereof. In other words, the Lactobacillus sakei or the metabolite thereof, when administrated to an subject, can delay cellular aging or oxidation, enhance mitochondrial activity, promote autophagy, prevent and/or reduce oxidative stress of the cells, increase GSH content, enhance GST activity, reduce MDA content, increase GST-RBC content, delay skin aging, increase skin collagen content, fade skin spots, reduce skin roughness, reduce skin fine lines and/or wrinkles, promote skin moisture retention capacity or any combination thereof. Therefore, the Lactobacillus sakei or the metabolite thereof is suitable for preparing a composition of delaying cellular aging or oxidation, enhancing mitochondrial activity, promoting autophagy, preventing and/or reducing oxidative stress of the cells, increasing GSH content, enhancing GST activity, reducing MDA content, increasing GST-RBC content, delaying skin aging, increasing skin collagen content, fading skin spots, reducing skin roughness, reducing skin fine lines and/or wrinkles, promoting skin moisture retention capacity or any combination thereof.

In some embodiments, a method for delaying cellular aging or oxidation, enhancing mitochondrial activity, promoting autophagy, preventing and/or reducing oxidative stress of the cells, increasing GSH content, enhancing GST activity, reducing MDA content, increasing GST-RBC content, delaying skin aging, increasing skin collagen content, fading skin spots, reducing skin roughness, reducing skin fine lines and/or wrinkles, promoting skin moisture retention capacity or any combination thereof includes administering to a subject in need thereof a composition including the Lactobacillus sakei or the metabolite thereof.

In some embodiments, the aforementioned subject may be a human.

In some embodiments, the aforementioned Lactobacillus sakei is included in the aforementioned composition in a form of bacterial powder.

In some embodiments, the aforementioned bacterial powder preparation procedure is to prepare a medium, sterilize and cool the medium, and then plant the aforementioned Lactobacillus sakei in the medium for fermentation culture. After the fermentation culture, ultrahigh-speed centrifugation is carried out to obtain fermentation-cultured strains. The fermentation-cultured strains are embedded or coated, and then the embedded or coated strains are lyophilized to obtain lyophilized strains. Subsequently, the lyophilized strains are ground and sieved, and then, the ground and sieved strains are packaged, and preserved in a refrigerator, to obtain bacterial powder.

In some embodiments, the aforementioned Lactobacillus sakei is included in the aforementioned composition in a form of live bacteria or dead bacteria.

In some embodiments, an effective dose of the aforementioned Lactobacillus sakei is 100 mg/day.

In some embodiments, the aforementioned composition may be a pharmaceutical composition, a food composition, a beverage composition or a nutritional supplement composition.

In some embodiments, when the aforementioned composition is the pharmaceutical composition, the pharmaceutical composition includes an effective dose of Lactobacillus sakei. The pharmaceutical composition may be manufactured into a dosage form suitable for being enterally, parenterally, orally or topically administrated by using a technology known to those skilled in the art.

In some embodiments, the enterally or orally administrated dosage form may be, but is not limited to, tablets, troches, lozenges, pills, capsules, dispersible powder, granules, solutions, suspensions, emulsions, syrup, elixirs, slurry or similar substances.

In some embodiments, the parenterally or topically administrated dosage form may be, but is not limited to, an injection (for example, a sterile aqueous solution or dispersion), sterile powder, an external preparation or similar substances.

In some embodiments, an administration mode of the injection may be, but is not limited to, intraperitoneal injection, subcutaneous injection, intraepidermal injection, intradermal injection, intramuscular injection, intravenous injection or intralesional injection.

In some embodiments, the pharmaceutical composition containing an effective dose of Lactobacillus sakei may further include a pharmaceutically acceptable carrier that is widely used in a pharmaceutical manufacturing technology. In some embodiments, the pharmaceutically acceptable carrier may be one or more of the following carriers: a solvent, a buffer, an emulsifier, a suspending agent, a decomposer, a disintegrating agent, a dispersing agent, a binding agent, an excipient, a stabilizing agent, a chelating agent, a diluent, a gelling agent, a preservative, a wetting agent, a lubricant, an absorption delaying agent, a liposome and similar substances. The type and quantity regarding the carrier selected fall within the scope of the professional quality and routine technology known to those skilled in the art. The solvent used as the pharmaceutically acceptable carrier may be water, normal saline, phosphate buffered saline (PBS) or aqueous solution containing alcohol.

In some embodiments, the pharmaceutical composition containing an effective dose of Lactobacillus sakei may be manufactured into an external preparation suitable for being topically administrated to the skin by using a technology known to those skilled in the art, including but not limited to: an emulsion, a gel, an ointment, a cream, a patch, a liniment, a powder, an aerosol, a spray, a lotion, a serum, a paste, a foam, a drop, a suspension, a salve, and a bandage.

In some embodiments, when the aforementioned pharmaceutical composition is the external preparation, the pharmaceutical composition may be made by mixing an effective dose of Lactobacillus sakei with a base known to those skilled in the art.

In some embodiments, the base may include one or more additives selected from the following additives: water, alcohols, glycol, hydrocarbons (such as petroleum, jelly and white petroleum), wax (such as paraffin and yellow wax), preserving agents, antioxidants, surfactants, absorption enhancers, stabilizing agents, gelling agents (such as Carbopol®974P, microcrystalline cellulose and carboxymethylcellulose), active agents, humectants, odor absorbers, fragrances, pH adjusting agents, chelating agents, emulsifiers, occlusive agents, emollients, thickeners, solubilizing agents, penetration enhancers, anti-irritants, colorants, propellants, etc. The selection and quantity regarding these additives fall within the scope of the professional quality and routine technology known to those skilled in the art.

In some embodiments, when the aforementioned composition is the food composition, the beverage composition or the nutritional supplement composition, the food composition, the beverage composition or the nutritional supplement composition includes an effective dose of Lactobacillus sakei. The food composition, the beverage composition or the nutritional supplement composition may be in a form of powder, granules, solutions, colloid or paste.

In some embodiments, the food composition, the beverage composition or the nutritional supplement composition containing the Lactobacillus sakei may be a food product or a food additive.

In some embodiments, the food composition, the beverage composition or the nutritional supplement composition containing the Lactobacillus sakei may be beverages, fermented foods, bakery products, health foods, dietary supplements or the like. In some embodiments, the food composition, the beverage composition or the nutritional supplement composition containing the Lactobacillus sakei may further include an adjuvant. For example, the adjuvant may be a maltodextrin, a malic acid, a sucralose, a citric acid, a fruit flavor, a honey flavor, a stevioside, or a combination thereof. The type and quantity regarding the adjuvant selected fall within the scope of the professional quality and routine technology known to those skilled in the art.

In some embodiments, the food composition, the beverage composition or the nutritional supplement composition containing the Lactobacillus sakei may be a flavoring, a sweetener, a flavor, a pH adjusting agent, an emulsifier, a colorant, a stabilizer, or the like.

Unless otherwise specified in the following examples, the experimental steps were carried out at room temperature (about 25° C.) and atmospheric pressure (1 atm).

### Example 1: Strain Identification

First, an isolated strain isolated from Lentinus edodes (purchased from HAND&SHARE INTERNATIONAL TRADE CO., LTD., origin: Oita Prefecture, Kyushu, Japan) underwent strain identification. After a polymerase chain reaction (PCR) product of the isolated strain was obtained by a PCR, sequencing was performed with Sanger sequencing to obtain a 16S ribosomal gene (16SrDNA) sequence (i.e., SEQ ID NO: 1). Subsequently, after alignment between the SEQ ID NO: 1 sequence with 16S ribosome gene (16SrDNA) sequences of other Lactobacillus sakei (as shown in Table I) on the website of the National Center of Biotechnology Information (NCBI), USA, it could be seen that the 16SrDNA sequence of this isolated strain had 100% identity with the 16SrDNA sequences of the other Lactobacillus sakei (as shown in Table I). Therefore, the isolated strain was named Lactobacillus sakei TCI147.

TABLE I

| | Percent |
|---|---|
| | Identity |
| | (Per. |
| Lactobacillus sakei | Ident) |
| Lactobacillus sakei strain MBEL1397 chromosome, complete | 100% |
| genome | |
| Lactobacillus sakei strain CBA3614 chromosome, complete | 100% |
| genome | |
| Lactobacillus sakei strain LZ217 chromosome, complete | 100% |
| genome | |
| Lactobacillus sakei strain ZFM220 chromosome, complete | 100% |
| genome | |
| Lactobacillus sakei strain ZFM225 chromosome, complete | 100% |
| genome | |

### Example 2: Preservation and Culture Experiments of Lactobacillus sakei TCI147

A. Preservation and Culture Materials:

1. MRSD medium, purchased from BD, with a product number of 288130.

B. Preservation and Culture Procedures:

1. The Lactobacillus sakei TCI147 isolated in Example 1 was cultured in the MRSD medium to obtain a bacterial solution. Then the bacterial solution and glycerin were mixed in a ratio of 4:1. After that, the mixture of the bacterial solution and the glycerin was preserved at −80° C.

2. The Lactobacillus sakei TCI147 was inoculated into the MRSD medium with 1% (v/v) of bacterial load (about 1×10⁴ CFU/mL), and was cultured at 37° C. for 24 h to form a Lactobacillus sakei TCI147 bacterial solution.

3. The Lactobacillus sakei TCI147 bacterial solution was centrifuged at 5,000 rpm for 5 min to obtain a supernatant. The supernatant was filtered with a 0.2 μm filter membrane. The obtained filtrate was a Lactobacillus sakei TCI147 sample (i.e., the Lactobacillus sakei TCI147 sample contained metabolites of the Lactobacillus sakei TCI147).

### Example 3

A. Materials and Instruments:

1. Cell line: Human blood was purified to obtain peripheral blood mononuclear cells (PBMCs), hereinafter referred to as PBMCs.

2. Cell medium: 4.2 X-VIVO™ 15 Serum-free Hematopoietic Cell Medium (purchased from Lonza, with a product number of 04-418Q), in which 5% (v/v) fetal bovine serum (FBS, purchased from Gibco, with a product number of 10437-028) and 1% (v/v) antibiotic (purchased from Gibco, with a product number of 15240-062) were added.

3. RNA extraction kit, purchased from Geneaid, with a product number of 301393.

4. SuperScript® III reverse transcriptase, purchased from Invitrogen, with a product number of 18080-044.

5. ABI StepOnePlus™ Real-Time PCR system, purchased from Thermo Fisher Scientific.

6. KAPA SYBR FAST qPCR Master Mix (2×) Kit, purchased from KAPA Biosystems, with a product number of KK4600.

B. Trial Procedures:

1. The PBMCs were inoculated into a 6-well culture plate at a density of 1×10⁶ cells per well, and were cultured at 37° C. for 24 h. Here, the PBMCs were divided into three trial groups: a blank group, a control group and an experimental group, respectively. Each group underwent trial in triplicate (that is, each group had three wells).

2. After the 24 h of culture, the 6-well culture plate was replaced with an experimental medium in each group, and culture was further performed at 37° C. for 48 h. The experimental medium in the blank group was a cell medium without any additives. The experimental medium in the control group was a cell medium containing a 0.5% (v/v) MRSD medium. The experimental medium in the experimental group was a cell medium containing the 0.5% (v/v) Lactobacillus sakei TCI147 sample prepared in Example 2.

3. The cultured blank group, control group and experimental group were centrifuged to remove the medium of each cultured group, and rinsing was performed with PBS.

4. After the rinsing, the cultured blank group, control group and experimental group were centrifuged to remove the PBS.

5. After the PBS was removed, a cell membrane of each of the PBMCs in each group was broken with an RB buffer of the RNA extraction kit to form a cell solution.

6. RNA in the cell solution of each group was extracted by using the RNA extraction kit.

7. 1,000 nanograms (ng) of the extracted RNA was used as a template in each group, and the extracted RNA was reverse-transcribed into corresponding cDNA by the SuperScript® III reverse transcriptase.

8. A quantitative real-time reverse transcription polymerase chain reaction was performed on the cDNA by using the ABI StepOnePlus™ Real-Time PCR system, with the KAPA SYBR FAST qPCR Master Mix (2λ) Kit and a combination primer of Table II, to observe expression of various target genes of HPEK-50 cells in the blank group and the experimental group and melting curves thereof. Instrument set conditions for the quantitative real-time reverse transcription polymerase chain reaction were as follows: reacting at 95° C. for 20 s, reacting at 95° C. for 3 s, and reacting at 60° C. for 30 s, and repeating for 40 cycles.

9. Relative expression of the target genes were determined by using a 2^{-ΔΔCt} method. The so-called relative expression was defined as a fold change of an RNA expression of the target gene in the experimental group or the blank group relative to that of the same gene in the blank group. In the 2^{-ΔΔCt} method, the fold change was calculated with a cycle threshold (Ct) of a TATA-box binding protein (TBP) gene as a cycle threshold of a reference gene of an internal control according to the following formula:

ΔCt=Ct_{target gene of experimental group/target gene of blank group}−Ct_{TBP}

ΔΔCt=ΔCt_{target gene of experimental group}−ΔCt_{target gene of blank group}

Fold change=2^{-ΔΔCt mean}

TABLE II

| Primer | Sequence | |
|---|---|---|
| Name | Number | Sequence |
| NADSYN1-F | SEQ ID NO: 2 | GGATGATACAGGACCTGACAAAG |
| NADSYN1-R | SEQ ID NO: 3 | CCGAGGCGTTGGTGATGAT |
| ATG8-F | SEQ ID NO: 4 | ACTCGCTGGAACACAGATGC |
| ATG8-R | SEQ ID NO: 5 | TCTGAGAGCCTGAGACCTTTT |
| FOXO-F | SEQ ID NO: 6 | CGGACAAACGGCTCACTCT |
| FOXO-R | SEQ ID NO: 7 | GGACCCGCATGAATCGACTAT |
| CCT6A-F | SEQ ID NO: 8 | ACACTCACTCAGATCAAAGATGC |
| CCT6A-R | SEQ ID NO: 9 | CCCTTTACACTGGGCTTATGTTT |
| CCT7-F | SEQ ID NO: 10 | TGTTTGAAGAGACCCAGATTGGA |
| CCT7-R | SEQ ID NO: 11 | ATGGCCCTCCTGACGATCAT |

10. Statistically significant differences between measurements of the blank group and other all groups, as well as between the control group and other all groups were statistically analyzed by a student t-test. In the figure, ‘*’ represents that the p value is less than 0.05 in comparison with the blank group, ‘**’ represents that the p value is less than 0.01 in comparison with the blank group, and ‘***’ represents that the p value is less than 0.001 in comparison with the blank group. In the figure, ‘#’ represents that the p value is less than 0.05 in comparison with the control group, ‘##’ represents that the p value is less than 0.01 in comparison with the control group, and ‘###’ represents that the p value is less than 0.001 in comparison with the control group.

Trial Results:

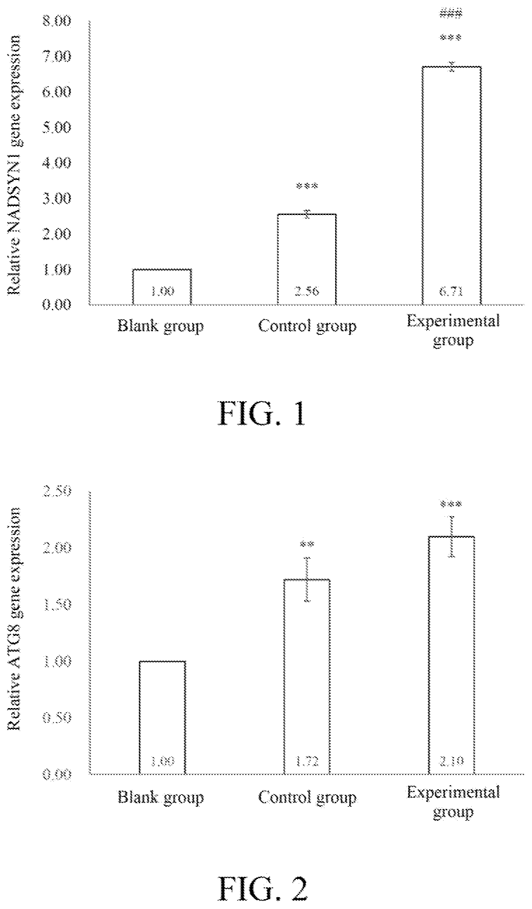

Refer to FIG. 1. The blank group was not treated with any additives, and therefore, the trial results of the blank group represented expression of the PBMCs under normal physiological metabolism. Here, in a case where a relative NADSYN1 gene expression of the blank group was set as 1, a relative NADSYN1 gene expression of the control group was 2.56, while a relative NADSYN1 gene expression of the experimental group was 6.71. That is, relative to that of the blank group, the relative NADSYN1 gene expression of the control group was significantly increased by about 156% after the MRSD medium was added to the PBMCs of the control group. Relative to that of the blank group, the relative NADSYN1 gene expression of the experimental group was significantly increased by about 571% after the Lactobacillus sakei TCI147 sample was added to the PBMCs of the experimental group. Relative to that of the control group, the relative NADSYN1 gene expression of the experimental group was significantly increased by about 415% after the Lactobacillus sakei TCI147 sample was added to the PBMCs of the experimental group.

It could be seen from the experimental results that the Lactobacillus sakei TCI147 sample could significantly increase the NADSYN1 gene expression, and the ability of the Lactobacillus sakei TCI147 sample to increase the NADSYN1 gene expression was significantly superior to that of the MRSD medium. An NADSYN1 gene is responsible for encoding NAD (+) synthetase 1, which can synthesize NAD (+). Literature pointed out that a sign of aging is a systematic decrease of NAD⁺ in multiple types of tissue. The decrease of NAD⁺ would lead to functional defects of nuclei and mitochondria, and lead to many age-related diseases. Increasing NAD (+) could significantly improve these age-related functional defects and eliminate many aging diseases. In other words, the Lactobacillus sakei TCI147 and/or the metabolites thereof in any embodiment could increase NAD (+) synthetase 1, increase the synthesis of NAD (+), thereby increasing intracellular NAD (+) content, enhancing functions of nuclei and mitochondria, enhancing mitochondrial activity, and delaying cellular aging.

Refer to FIG. 2. The blank group was not treated with any additives, and therefore, the trial results of the blank group represented expression of the PBMCs under normal physiological metabolism. Here, in a case where a relative ATG8 gene expression of the blank group was set as 1, a relative ATG8 gene expression of the control group was 1.72, while a relative ATG8 gene expression of the experimental group was 2.10. That is, relative to that of the blank group, the relative ATG8 gene expression of the control group was significantly increased by about 72% after the MRSD medium was added to the PBMCs of the control group. Relative to that of the blank group, the relative ATG8 gene expression of the experimental group was significantly increased by about 110% after the Lactobacillus sakei TCI147 sample was added to the PBMCs of the experimental group. Relative to that of the control group, the relative ATG8 gene expression of the experimental group was significantly increased by about 38% after the Lactobacillus sakei TCI147 sample was added to the PBMCs of the experimental group.

It could be seen from the experimental results that the Lactobacillus sakei TCI147 sample could significantly increase the ATG8 gene expression, and the ability of the Lactobacillus sakei TCI147 sample to increase the ATG8 gene expression was significantly superior to that of the MRSD medium. An ATG8 gene is responsible for encoding an autophagy-associated protein 8. Autophagy is an anti-aging mechanism of cells. The cells get enough nutrients, by breaking down their own unnecessary and dysfunctional structures and organelles, to survive damage of stress. The autophagy-associated protein 8 is one of key molecular components involved in the autophagy, and is a protein required for autophagosome membrane formation. With age, the autophagy efficiency of old cells decreases, thereby leading to cell damage and accumulation of senescent cells. In other words, the Lactobacillus sakei TCI147 and/or the metabolites thereof could increase autophagy-associated protein 8, thereby promoting autophagy, increasing the efficiency of autophagy, removing waste, avoiding cell damage and accumulation of senescent cells, to delay cellular aging. The Lactobacillus sakei TCI147 and/or the metabolites thereof in any embodiment could resist external stress and internal damage by maintaining normal and efficient autophagy, thereby achieving an anti-aging effect.

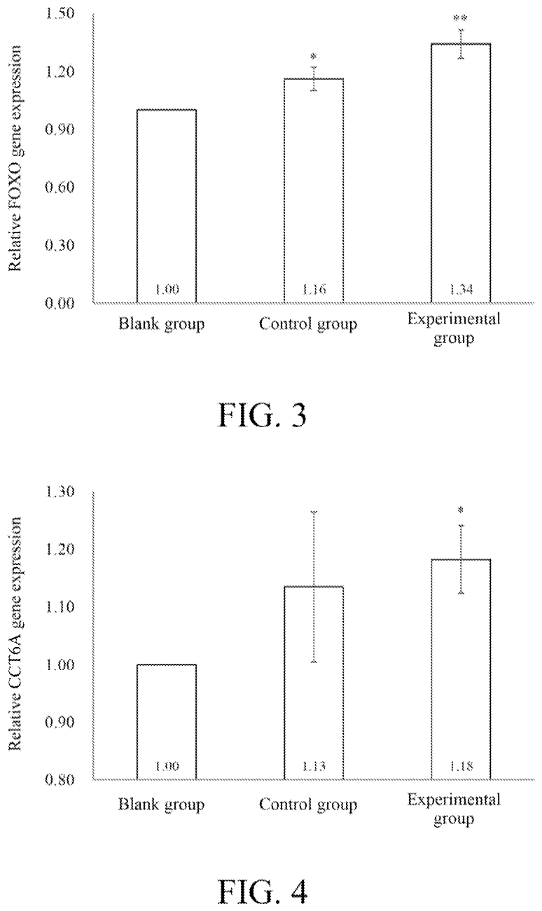

Refer to FIG. 3. The blank group was not treated with any additives, and therefore, the trial results of the blank group represented expression of the PBMCs under normal physiological metabolism. Here, in a case where a relative FOXO gene expression of the blank group was set as 1, a relative FOXO gene expression of the control group was 1.16, while a relative FOXO gene expression of the experimental group was 1.34. That is, relative to that of the blank group, the relative FOXO gene expression of the control group was significantly increased by about 16% after the MRSD medium was added to the PBMCs of the control group. Relative to that of the blank group, the relative FOXO gene expression of the experimental group was significantly increased by about 34% after the Lactobacillus sakei TCI147 sample was added to the PBMCs of the experimental group. Relative to that of the control group, the relative FOXO gene expression of the experimental group was significantly increased by about 18% after the Lactobacillus sakei TCI147 sample was added to the PBMCs in the experimental group.

It could be seen from the experimental results that the Lactobacillus sakei TCI147 sample could significantly increase the FOXO gene expression, and the ability of the Lactobacillus sakei TCI147 sample to increase the FOXO gene expression was significantly superior to that of the MRSD medium. An FOXO gene is responsible for encoding an FOXO protein, which has been proved to be involved in regulation of the autophagy, to maintain the balance between protein synthesis and degradation, thereby alleviating hypofunction when the cells age. The FOXO protein has been proved by literature to be one of genes that exhibited a consistent association with longevity, and is an important determinant of aging and longevity. In other words, the Lactobacillus sakei TCI147 and/or the metabolites thereof in any embodiment could increase FOXO protein, thereby regulating autophagy, to maintain the balance between protein synthesis and degradation. The Lactobacillus sakei TCI147 and/or the metabolites thereof in any embodiment could alleviate hypofunction when the cells age, so as to delay cellular aging, thereby achieving an anti-aging effect.

Refer to FIG. 4. The blank group was not treated with any additives, and therefore, the trial results of the blank group represented expression of the PBMCs under normal physiological metabolism. Here, in a case where a relative CCT6A gene expression of the blank group was set as 1, a relative CCT6A gene expression of the control group was 1.13, while a relative CCT6A gene expression of the experimental group was 1.18. That is, relative to that of the blank group, the relative CCT6A gene expression of the control group was increased by about 13% after the MRSD medium was added to the PBMCs of the control group. Relative to that of the blank group, the relative CCT6A gene expression of the experimental group was significantly increased by about 18% after the Lactobacillus sakei TCI147 sample was added to the PBMCs in the experimental group. Relative to that of the control group, the relative CCT6A gene expression of the experimental group was significantly increased by about 5% after the Lactobacillus sakei TCI147 sample was added to the PBMCs of the experimental group.

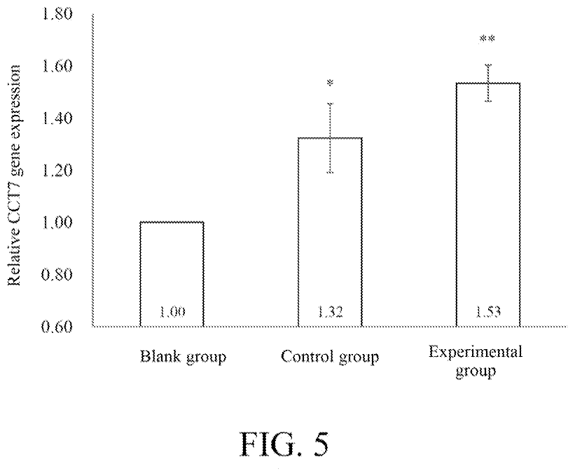

Refer to FIG. 5. The blank group was not treated with any additives, and therefore, the trial results of the blank group represented expression of the PBMCs under normal physiological metabolism. Here, in a case where a relative CCT7 gene expression of the blank group was set as 1, a relative CCT7 gene expression of the control group was 1.32, while a relative CCT7 gene expression of the experimental group was 1.53. That is, relative to that of the blank group, the relative CCT7 gene expression of the control group was significantly increased by about 32% after the MRSD medium was added to the PBMCs in the control group. Relative to that of the blank group, the relative CCT7 gene expression of the experimental group was significantly increased by about 53% after the Lactobacillus sakei TCI147 sample was added to the PBMCs in the experimental group. Relative to that of the control group, the relative CCT7 gene expression of the experimental group was significantly increased by about 21% after the Lactobacillus sakei TCI147 sample was added to the PBMCs in the experimental group.

It could be seen from the experimental results that the Lactobacillus sakei TCI147 sample could significantly increase the CCT6A and CCT7 gene expressions, and the ability of the Lactobacillus sakei TCI147 sample to increase the CCT6A and CCT7 gene expressions was significantly superior to that of the MRSD medium. CCT6A and CCT7 genes are responsible for encoding chaperone proteins. Literature pointed out that the chaperone protein could help protein folding and participates in regulation of telomere maintenance, to promote cell rejuvenation. In other words, the Lactobacillus sakei TCI147 and/or the metabolites thereof in any embodiment could increase chaperone proteins, thereby helping protein folding, improving telomere maintenance, to promote cell rejuvenation, thus delaying cellular aging.

### Example 4

A. Materials and Instruments:

1. Cell line: Human liver cells, purchased from the American Type Culture Collection (ATCC), with a cell number of HB-8065, hereinafter referred to as HepG2 cells.

2. Cell medium: DMEM (purchased from Gibco, with a product number of 11965-092), in which 5% (v/v) fetal bovine serum (FBS, purchased from Gibco, with a product number of 10437-028) and 1% (v/v) antibiotic (purchased from Gibco, with a product number of 15240-062) were added.

3. GST activity assay kit, purchased from abcam. This kit included a GST assay buffer, a GST substrate, a GST positive control and glutathione.

4. GST reaction mixture: Prepared from the GST assay buffer and GST substrate of the aforementioned kit. A preparation ratio of the GST assay buffer to the GST substrate was 49:1.

5. Test sample of a positive control: Prepared from the GST assay buffer and the GST positive control of the aforementioned kit. A preparation volume of the GST assay buffer to the GST positive control was 198 μL:2 μL. The dilution ratio was 100×dilution.

6. Trypsin, purchased from Gibco, with a product number of 15400-054.

7. Test instrument: ELISA reader, purchased from BioTek (USA).

B. Trial Procedures:

1. The HepG2 cells were inoculated into a 6-well culture plate at a density of 2×10⁶ cells per well, and was cultured at 37° C. for 24 h. Here, the cells were divided into three trial groups: a blank group, a control group and an experimental group, respectively. Each group underwent trial in triplicate (that is, each group had three wells).

2. After the 24 h of culture, the 6-well culture plate was replaced with an experimental medium in each group, and culture was further performed at 37° C. for 24 h. The experimental medium in the blank group was a cell medium without additives. The experimental medium in the control group was a cell medium containing a 0.5% (v/v) MRSD medium. The experimental medium in the experimental group was a cell medium containing the 0.5% (v/v) Lactobacillus sakei TCI147 sample prepared in Example 2.

3. After the 24 h of culture, the experimental medium of each cultured group was removed, and rinsing was performed twice with PBS.

4. After the rinsing, 200 μL of trypsin was added into each well for a reaction for 3 min. After the reaction, 6 mL of the cell medium was added to terminate the reaction. Then the suspended cells and the cell medium in each well were collected into corresponding centrifuge tubes.

5. After each centrifuge tube was centrifuged to precipitate the cells, a supernatant in the centrifuge tube in each group was removed, then the precipitated cells were rinsed with the PBS and suspended twice to obtain a cell suspension redissolved with the 100 μL of the GST assay buffer, and well mixing was performed.

6. After each centrifuge tube underwent high-speed centrifugation, the supernatant in each centrifuge tube was collected to obtain test samples of each group.

7. 50 μL of the test sample of each group, 50 μL of the test sample of the positive control, and 50 μL of the GST assay buffer were added into each well in a 96-well culture plate.

8. 5 μL of glutathione was added into each well.

9. 50 μL of the GST reaction mixture was added into each well and well mixed, and then was placed at 37° C. for a reaction for 5 min.

10. An absorbance value at 340 nm (OD₃₄₀ value) per well was measured by using the ELISA reader.

11. Relative glutathione S-transferase activity (relative GST activity) of all groups was calculated according to the following formula: relative GST activity (%)=(OD₃₄₀ value of each group/OD₃₄₀ value of blank group)×100%.

12. Statistically significant differences between measurements of the blank group and other all groups were statistically analyzed by a student t-test. In the figure, ‘*’ represents that the p value is less than 0.05 in comparison with the blank group, ‘**’ represents that the p value is less than 0.01 in comparison with the blank group, and ‘***’ represents that the p value is less than 0.001 in comparison with the blank group.

C. Trial Results:

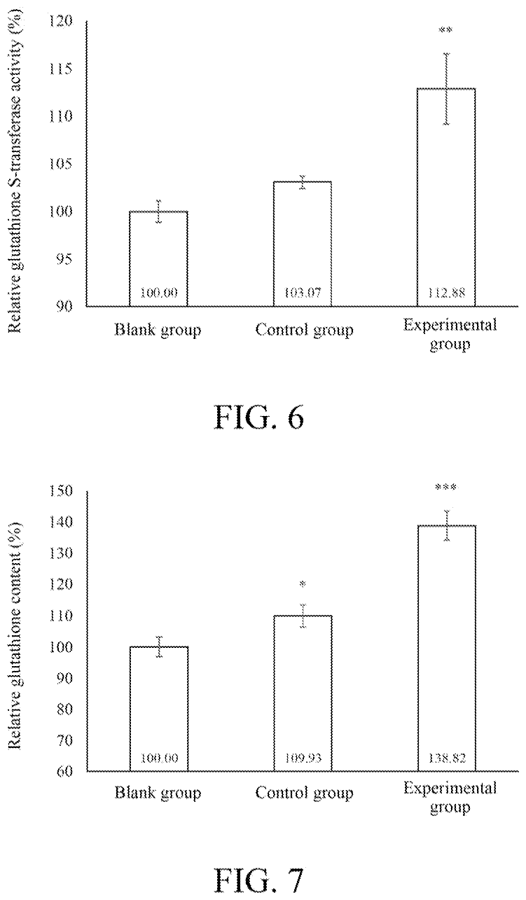

Refer to FIG. 6. The HepG2 cells in the blank group were present alone, and were not treated with any additives, and therefore, the trial results of the blank group represented expression of the HepG2 cells under normal physiological metabolism. Here, in a case where the relative GST activity of the blank group was set as 100%, the relative GST activity of the control group was 103.07%, while the relative GST activity of the experimental group was 112.88%. That is, relative to that of the blank group, the relative GST activity of the control group was enhanced by about 3% after the MRSD medium was added to the cells in the control group. Relative to that of the blank group, the relative GST activity of the experimental group was significantly enhanced by about 13% after the Lactobacillus sakei TCI147 sample was added to the cells in the experimental group. Relative to that of the control group, the relative GST activity of the experimental group was enhanced by about 10%.

It could be seen from the experimental results that the Lactobacillus sakei TCI147 sample could significantly enhance the GST activity, and enhance the GST activity of the liver cells, and the ability of the Lactobacillus sakei TCI147 sample to enhance the GST activity was significantly superior to that of the MRSD medium. Glutathione S-transferases (GST) are key enzymes of a glutathione binding reaction, which can catalyze an initial step of the glutathione binding reaction. The glutathione S-transferase has antioxidant and detoxification functions, so as to protect an organism. In other words, the Lactobacillus sakei TCI147 and/or the metabolites thereof in any embodiment could increase glutathione S-transferase content and enhance glutathione S-transferase activity, thereby improving antioxidant capacity and delaying cellular oxidation. The Lactobacillus sakei TCI147 and/or the metabolites thereof in any embodiment could increase glutathione S-transferase content of the liver cells, enhance glutathione S-transferase activity of the liver cells, thereby improving antioxidant capacity of the liver cells and delaying oxidation of the liver cells. The Lactobacillus sakei TCI147 and/or the metabolites thereof in any embodiment help to resist intracellular oxidative stress, prevented and/or reduced oxidative stress of the cells, and delayed cellular aging caused by oxidative stress. The Lactobacillus sakei TCI147 and/or the metabolites thereof in any embodiment help to resist intracellular oxidative stress of the liver cells, prevented and/or reduced oxidative stress of the liver cells, and delayed aging of the liver cells caused by oxidative stress.

### Example 5

A. Materials and Instruments:

1. Cell line: Human blood was purified to obtain peripheral blood mononuclear cells (PBMCs), hereinafter referred to as PBMCs.

2. Cell medium: 4.2 X-VIVO™ 15 Serum-free Hematopoietic Cell Medium (purchased from Lonza, with a product number of 04-418Q), in which 5% (v/v) fetal bovine serum (FBS, purchased from Gibco, with a product number of 10437-028) and 1% (v/v) antibiotic (purchased from Gibco, with a product number of 15240-062) were added.

3. GSH detection reagent, purchased from abcam, with a product number of Ab112132.

4. Flow cytometer, purchased from BD company, with a model of Accuri™ C6 Plus.

B. Trial Procedures:

1. The PBMCs were inoculated into a 6-well culture plate at a density of 2×10⁵ cells per well, and were cultured at 37° C. for 24 h. Here, the cells were divided into three trial groups: a blank group, a control group and an experimental group, respectively. Each group underwent trial in triplicate (that is, each group had three wells). The experimental medium in the blank group was a cell medium without additives. The experimental medium in the control group was a cell medium containing a 0.5% (v/v) MRSD medium. The experimental medium in the experimental group was a cell medium containing the 0.5% (v/v) Lactobacillus sakei TCI147 sample prepared in Example 2.

2. After the 24 h of culture, the GSH detection reagent (a staining ratio was 1:1,000) was added to each well to react for 15 min.

3. The cells, the medium and the reagent in each well were collected into corresponding centrifuge tubes, the cells were rinsed with the PBS, and the cells were then resuspended with the PBS. Subsequently, analysis was performed with the flow cytometer and green fluorescent signal quantification was performed on each group. Here, a fluorescent signal value of each group was obtained.

4. Relative glutathione content (relative GSH content) of all groups was calculated according to the following formula: relative GSH content (%)=(fluorescent signal value of each group/fluorescent signal value of blank group)×100%.

5. Statistically significant differences between measurements of the blank group and other all groups were statistically analyzed by a student t-test. In the figure, ‘*’ represents that the p value is less than 0.05 in comparison with the blank group, ‘**’ represents that the p value is less than 0.01 in comparison with the blank group, and ‘***’ represents that the p value is less than 0.001 in comparison with the blank group.

C. Trial Results:

Refer to FIG. 7. The cells in the blank group were present alone, and were not treated with any additives, and therefore, the trial results of the blank group represented expression of the cells under normal physiological metabolism. Here, in a case where the relative GSH content of the blank group was set as 100%, the relative GSH content of the control group was 109.93%, while the relative GSH content of the experimental group was 138.82%. That is, relative to that of the blank group, the relative GSH content of the control group was significantly increased by about 10% after the MRSD medium was added to the cells in the control group. Relative to that of the blank group, the relative GSH content of the experimental group was significantly increased by about 39% after the Lactobacillus sakei TCI147 sample was added to the cells in the experimental group. Relative to that of the control group, the relative GSH content of the experimental group was increased by about 29%.

It could be seen from the experimental results that the Lactobacillus sakei TCI147 sample could significantly increase the GSH content, and the ability of the Lactobacillus sakei TCI147 sample to increase the GSH content was significantly superior to that of the MRSD medium. Glutathione (GSH) can scavenge free radicals and is a most abundant antioxidant in human bodies. Glutathione (GSH) is also an important antidote in the human body, which can help metabolism and detoxification of the cells. In other words, the Lactobacillus sakei TCI147 and/or the metabolites thereof in any embodiment could increase glutathione content, to remove free radicals in the body, and could promote metabolism and detoxification in the body. The Lactobacillus sakei TCI147 and/or the metabolites thereof in any embodiment help to enhance intracellular redox capacity, resist intracellular oxidative stress, and reduce cellular aging caused by oxidative stress. The Lactobacillus sakei TCI147 and/or the metabolites thereof in any embodiment have antioxidant capacity.

### Example 6

A. Materials and Instruments:

1. Cell line: Human retinal pigment epithelial cells, purchased from the American Type Culture Collection (ATCC), with a cell number of CRL-2302, hereinafter referred to as ARPE-19 cells.

2. Cell medium: Dulbecco's Modified Eagle Medium/Nutrient Mixture F-12 (DMEM/F-12), purchased from Gibco, with a product number of 12500-062.

3. Trypsin, purchased from abcam, with a product number of Ab112132.

4. ActinRed™ 555 ReadyProbes™ Reagent, purchased from Thermo, with a product number of R37112.

5. Hoechst, purchased from Thermo, with a product number of 62249.

6. 100 mM H₂O₂: prepared from H₂O₂ (purchased from Sigma, with a product number of 1.08600) and DPBS.

7. Flow cytometer, purchased from BD company, with a model of Accuri™ C6 Plus.

8. Fluorescent microscope, purchased from ZEISS, with a model of Vert.A1.

B. Trial Procedures:

1. The ARPE-19 cells were inoculated into a 6-well culture plate at a density of 2×10⁵ cells per well, and were cultured at 37° C. for 24 h. Here, the cells were divided into four trial groups: a blank group, a H₂O₂ group, a control group and an experimental group, respectively. Each group underwent trial in triplicate (that is, each group had three wells).

2. After the 24 h of culture, a 0.5% (v/v) MRSD medium was added to the control group, and the 0.5% (v/v) Lactobacillus sakei TCI147 sample prepared in Example 2 was added to the experimental group, and each group was cultured at 37° C. for 1 h.

3. After the 1 h of culture, 100 mM H₂O₂ was added to each of the H₂O₂ group, the control group and the experimental group to reach a final concentration of 100 μM. Subsequently, each group was left for a reaction at 37° C. for 2 h.

4. After the 2 h of reaction, a supernatant of each group was removed, a fresh cell medium was added, and each group was cultured at 37° C. for 3 d to 7 d. The number of days of culture depends on a cell state observed under the microscope.

5. After the culture, the cell medium of each cultured group was removed, and rinsing was performed twice with PBS.

6. After the rinsing, trypsin was added into each well for a reaction for 3 min. After the reaction, the cell medium was added to terminate the reaction. Then the suspended cells and the cell medium in each well were collected into corresponding centrifuge tubes.

7. Some of the cells were taken out from each centrifuge tube and were inoculated into a 24-well culture plate, and were cultured at 37° C. for 48 h. Remaining cells in each centrifuge tube were rinsed with the PBS, and then the rinsed cells were resuspended with the PBS. Subsequently, the cell volume of each group was selected and analyzed with a flow cytometer, and a cell size of each group was obtained.

8. After the 48 h of culture at 37° C., 1 drop of ActinRed™ 555 ReadyProbes™ reagent and Hoechst (1:20,000 dilution) were added to each well in the 24-well culture plate for staining in dark for 15 min.

9. After the staining, observation was performed with the fluorescent microscope in dark and staining results of the cells in each group were photographed. In the staining results, green fluorescence represented signals of cytoskeletons, while blue fluorescence represented signals of nuclei.

10. Statistically significant differences between measurements of the H₂O₂ group and other all groups were statistically analyzed by a student t-test. In the figure, ‘#’ represents that the p value is less than 0.05 in comparison with the H₂O₂ group, ‘##’ represents that the p value is less than 0.01 in comparison with the H₂O₂ group, and ‘###’ represents that the p value is less than 0.001 in comparison with the H₂O₂ group.

C. Trial Results:

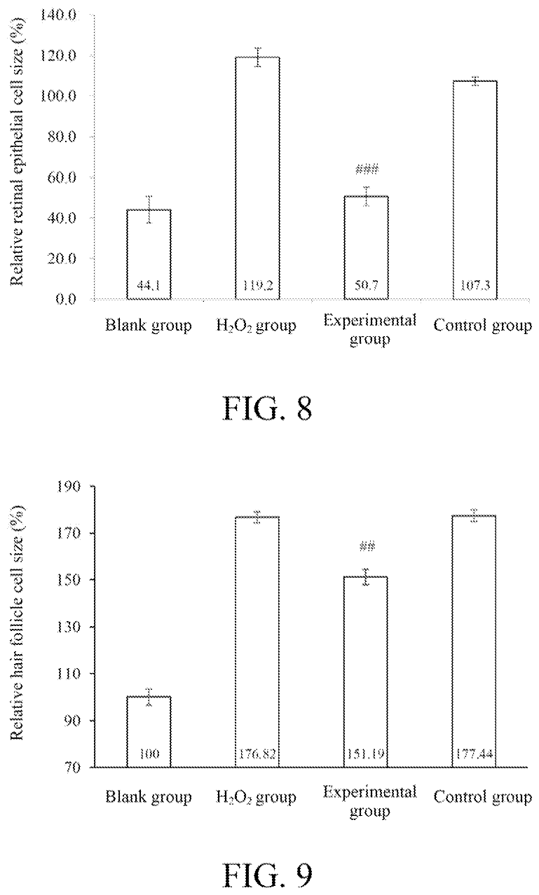

Refer to FIG. 8. The cells in the blank group were present alone, and were neither treated with any additives nor stimulated with H₂O₂, and therefore, the trial results of the blank group represented expression of the cells under normal physiological metabolism. Here, a relative retinal epithelial cell size of the blank group was 44.1%, a relative retinal epithelial cell size of the H₂O₂ group was 119.2%, a relative retinal epithelial cell size of the control group was 107.3%, and a relative retinal epithelial cell size of the experimental group was 50.7%. That is, relative to that of the blank group, the relative retinal epithelial cell size of the H₂O₂ group was significantly increased by about 170% after the cells in the H₂O₂ group were stimulated with the H₂O₂. Relative to that of the H₂O₂ group, the relative retinal epithelial cell size of the experimental group was significantly reduced by about 57% after the Lactobacillus sakei TCI147 sample was added to the cells in the experimental group and the cells were stimulated with the H₂O₂. Relative to that of the H₂O₂ group, the relative retinal epithelial cell size of the control group was decreased by about 10% after the MRSD medium was added to the cells in the control group and the cells were stimulated with the H₂O₂.

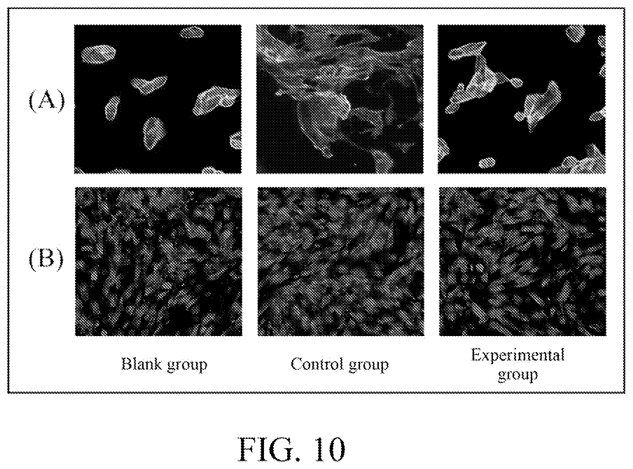

Refer to FIG. 10(A). Green and blue fluorescent signals of the cells in the blank group were intensive and dense, which indicated that the nuclei and surrounding cytoskeletons of the cells in the blank group were intensive and dense, which was expression of the cells under normal physiological metabolism. Relative to those in the blank group, green and blue fluorescent signals of the cells in the experimental group were intensive and dense as well, which indicated that the nuclei and surrounding cytoskeletons of the cells in the experimental group were intensive and dense, which were almost the same as those in the blank group. Relative to those in the blank group, green and blue signals of the cells in the control group were scattered and the green signals were looser, which indicated that nuclei and surrounding cytoskeletons of the cells in the control group were looser, and the cells were in an expanded state.

It could be seen from the experimental results that the Lactobacillus sakei TCI147 sample could significantly reduce a retinal epithelial cell size, and significantly reduce cell expansion, making the nuclei and cytoskeletons intensive and dense, which were similar to the expression of the cells under the normal physiological metabolism, and the ability of the Lactobacillus sakei TCI147 sample to reduce the retinal epithelial cell size was significantly superior to that of the MRSD medium. In this example, the oxidative stress was induced by adding the H₂O₂, which accelerated cellular aging. After aging, the cells would be prone to disintegrate due to abnormal expansion. Here, whether the sample could protect the cells from aging caused by the oxidative stress is determined by observing the shape and size of the cells and observing conditions of their nuclei and cytoskeletons by staining. In other words, the Lactobacillus sakei TCI147 and/or the metabolites thereof in any embodiment could reduce and/or prevent cellular aging caused by oxidative stress, and enhance resistance of the cells to oxidative stress. The Lactobacillus sakei TCI147 and/or the metabolites thereof in any embodiment could reduce and/or prevent aging of retinal epithelial cells caused by oxidative stress, so as to achieve an eye protection effect.

### Example 7

A. Materials and Instruments:

1. Cell line: Human hair follicle dermal papilla cells, purchased from PromoCell, with a cell number of C-12071, hereinafter referred to as HFDPC cells.

2. Cell medium: Follicle Dermal Papilla Cell Growth Medium, purchased from PromoCell, with a product number of C-26501.

3. Trypsin, purchased from abcam, with a product number of Ab112132.

4. ActinRed™ 555 ReadyProbes™ Reagent, purchased from Thermo, with a product number of R37112.

5. Hoechst, purchased from Thermo, with a product number of 62249.

6. 100 mM H₂O₂: prepared from H₂O₂ (purchased from Sigma, with a product number of 1.08600) and DPBS.

7. Flow cytometer, purchased from BD company, with a model of Accuri™ C6 Plus.

8. Fluorescent microscope, purchased from ZEISS, with a model of Vert.A1.

B. Trial Procedures:

1. The HFDPC cells were inoculated into a 6-well culture plate at a density of 2×10⁵ cells per well, and were cultured at 37° C. for 24 h. Here, the cells were divided into four trial groups: a blank group, a H₂O₂ group, a control group and an experimental group, respectively. Each group underwent trial in triplicate (that is, each group had three wells).

2. After the 24 h of culture, a 0.5% (v/v) MRSD medium was added to the control group, and the 0.5% (v/v) Lactobacillus sakei TCI147 sample prepared in Example 2 was added to the experimental group, and each group was cultured at 37° C. for 1 h.

3. After the 1 h of culture, 100 mM H₂O₂ was added to each of the H₂O₂ group, the control group and the experimental group to reach a final concentration of 30 μM. Subsequently, each group was left for a reaction at 37° C. for 2 h.

4. After the 2 h of reaction, a supernatant of each group was removed, a fresh cell medium was added, and each group was cultured at 37° C. for 3 d to 7 d. The number of days of culture depends on a cell state observed under the microscope.

5. After the culture, the cell medium of each cultured group was removed, and rinsing was performed twice with PBS.

6. After the rinsing, trypsin was added into each well for a reaction for 3 min. After the reaction, the cell medium was added to terminate the reaction. Then the suspended cells and the cell medium in each well were collected into corresponding centrifuge tubes.

7. Some of the cells were taken out from each centrifuge tube and were inoculated into a 24-well culture plate, and were cultured at 37° C. for 48 h. Remaining cells in each centrifuge tube were rinsed with the PBS, and then the rinsed cells were resuspended with the PBS. Subsequently, the cell volume of each group was selected and analyzed with a flow cytometer, and a cell size of each group was obtained.

8. The relative cell sizes of all groups were calculated according to the following formula: relative hair follicle cell size (%)=(cell size of each group/cell size of blank group)×100%.

9. After the 48 h of culture at 37° C., 1 drop of ActinRed™ 555 ReadyProbes™ reagent and Hoechst (1:20,000 dilution) were added to each well in the 24-well culture plate for staining in dark for 15 min.

10. After the staining, observation was performed with the fluorescent microscope in dark and staining results of the cells in each group were photographed. In the staining results, red fluorescence represented signals of cytoskeletons, while blue fluorescence represented signals of nuclei.

11. Statistically significant differences between measurements of the H₂O₂ group and other all groups were statistically analyzed by a student t-test. In the figure, ‘#’ represents that the p value is less than 0.05 in comparison with the H₂O₂ group, ‘##’ represents that the p value is less than 0.01 in comparison with the H₂O₂ group, and ‘###’ represents that the p value is less than 0.001 in comparison with the H₂O₂ group.

C. Trial Results:

Refer to FIG. 9. The cells in the blank group were present alone, and were neither treated with any additives nor stimulated with H₂O₂, and therefore, the trial results of the blank group represented expression of the cells under normal physiological metabolism. Here, a relative hair follicle cell size of the blank group was 100%, a relative hair follicle cell size of the H₂O₂ group was 176.82%, a relative hair follicle cell size of the control group was 177.44%, and a relative hair follicle cell size of the experimental group was 151.19%. That is, relative to that of the blank group, the relative hair follicle cell size of the H₂O₂ group was significantly increased by about 77% after the cells in the H₂O₂ group were stimulated with the H₂O₂. Relative to that of the H₂O₂ group, the relative hair follicle cell size of the experimental group was significantly reduced by about 14% after the Lactobacillus sakei TCI147 sample was added to the cells in the experimental group and the cells were stimulated with the H₂O₂. Relative to that of the H₂O₂ group, the relative hair follicle cell size of the control group was increased by about 0.4% instead of a decrease after the MRSD medium was added to the cells in the control group and the cells were stimulated with the H₂O₂.

Refer to FIG. 10(B). Red and blue fluorescent signals of the cells in the blank group were intensive and dense, which indicated that the nuclei and surrounding cytoskeletons of the cells in the blank group were intensive and dense, which was expression of the cells under normal physiological metabolism. Relative to those in the blank group, red and blue fluorescent signals of the cells in the experimental group were intensive and dense as well, which indicated that the nuclei and surrounding cytoskeletons of the cells in the experimental group were intensive and dense, which were almost the same as those in the blank group. Relative to those in the blank group, red and blue signals of the cells in the control group were scattered and the red signals were looser, which indicated that nuclei and surrounding cytoskeletons of the cells in the control group were looser, and the cells were in an expanded state.

It could be seen from the experimental results that the Lactobacillus sakei TCI147 sample could significantly reduce a hair follicle cell size, and significantly reduce cell expansion, making the nuclei and cytoskeletons intensive and dense, which were similar to the expression of the cells under the normal physiological metabolism, and the ability of the Lactobacillus sakei TCI147 sample to reduce the hair follicle cell size was significantly superior to that of the MRSD medium. In this example, the oxidative stress was induced by adding the H₂O₂, which accelerated cellular aging. After aging, the cells would be prone to disintegrate due to abnormal expansion. Here, whether the sample could protect the cells from aging caused by the oxidative stress is determined by observing the shape and size of the cells and observing conditions of their nuclei and cytoskeletons by staining. In other words, the Lactobacillus sakei TCI147 and/or the metabolites thereof in any embodiment could reduce and/or prevent cellular aging caused by oxidative stress, and enhance resistance of the cells to oxidative stress. The Lactobacillus sakei TCI147 and/or the metabolites thereof in any embodiment could reduce and/or prevent aging of the hair follicle cells caused by oxidative stress, so as to achieve an effect of preventing and/or delaying hair loss.

### Example 8

A. Materials and Instruments:

1. Cell line: Human skin fibroblasts, purchased from ATCC, with a cell number of CRL-1881, hereinafter referred to as CCD-966Sk cells.

2. Cell medium: Minimum essential medium (MEM, purchased from Gibco, with a product number of 11095080), in which 10% (v/v) FBS (purchased from Gibco, with a product number of 10437-028), 1% (v/v) antibiotic (purchased from Gibco, with a product number of 15140122) and 1 mM sodium pyruvate (purchased from Gibco, with a product number of 11360-070) were added.

3. Trypsin, purchased from Gibco, with a product number of 15400-054.

4. Soluble collagen assay kit, purchased from Biocolor, with a product number of S1000.

5. Test instrument: ELISA reader, purchased from BioTek (USA).

B. Trial Procedures:

1. The CCD-966Sk cells were inoculated into a 24-well culture plate at a density of 2×10⁴ cells per well, and were cultured at 37° C. for 24 h. Here, the cells were divided into three trial groups: a blank group, a control group and an experimental group, respectively. Each group underwent trial in triplicate (that is, each group had three wells).

2. After the 24 h of culture, rinsing was performed once with PBS, and the 24-well culture plate in each group was replaced with an experimental medium. The experimental medium in the blank group was a cell medium without additives. The experimental medium in the control group was a cell medium containing a 0.5% (v/v) MRSD medium. The experimental medium in the experimental group was a cell medium containing the 0.5% (v/v) Lactobacillus sakei TCI147 sample prepared in Example 2. Subsequently, culture was further performed at 37° C. for 48 h.

3. The experimental medium was taken out from each well of each cultured group, and the collagen secretion content of the CCD-966Sk cells in each group was determined by using the soluble collagen assay kit. Here, after the experimental medium taken out from each group was treated according to trial procedures provided by the soluble collagen assay kit, an absorbance value at 555 nm (OD₅₅₅ value) per well was measured by using the ELISA reader.

4. The relative collagen secretion content of all groups was calculated according to the following formula: relative collagen secretion content (%)=(OD₅₅₅ value of each group/OD₅₅₅ value of blank group)×100%.

5. Statistically significant differences between measurements of the blank group and other all groups, as well as between the control group and other all groups were statistically analyzed by a student t-test. In the figure, ‘*’ represents that the p value is less than 0.05 in comparison with the blank group, ‘**’ represents that the p value is less than 0.01 in comparison with the blank group, and ‘***’ represents that the p value is less than 0.001 in comparison with the blank group. In the figure, ‘#’ represents that the p value is less than 0.05 in comparison with the control group, ‘##’ represents that the p value is less than 0.01 in comparison with the control group, and ‘###’ represents that the p value is less than 0.001 in comparison with the control group.

C. Trial Results:

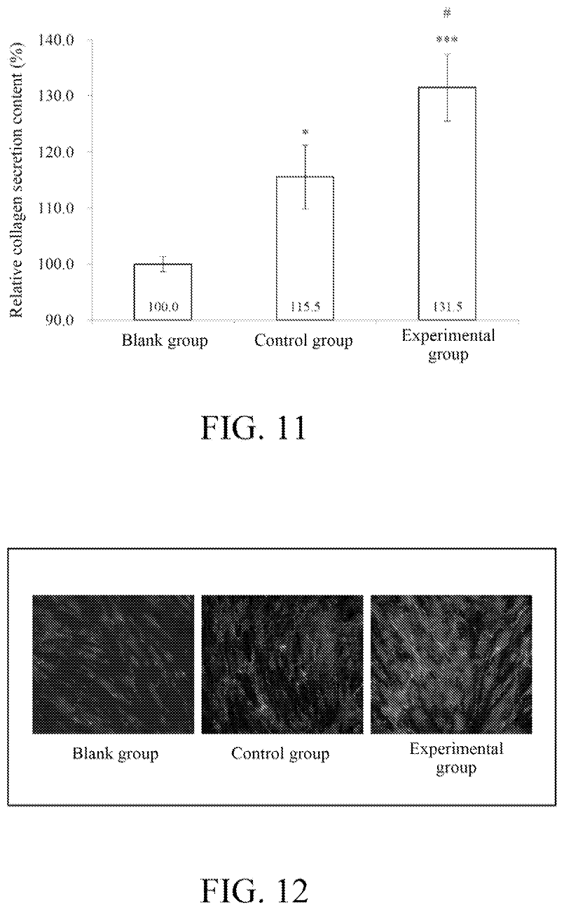

Refer to FIG. 11. The cells in the blank group were present alone, and were not treated with any additives, and therefore, the trial results of the blank group represented expression of the cells under normal physiological metabolism. Here, in a case where the relative collagen secretion content of the blank group was set as 100%, the relative collagen secretion content of the control group was 115.5%, while the relative collagen secretion content of the experimental group was 131.5%. That is, relative to that of the blank group, the relative collagen secretion content of the control group was significantly increased by about 15.5% after the CCD-966Sk cells in the control group were treated with the MRSD medium. Relative to that of the blank group, the relative collagen secretion content of the experimental group was significantly increased by about 31.5% after the CCD-966Sk cells in the experimental group were treated with the Lactobacillus sakei TCI147 sample. Relative to that of the control group, the relative collagen secretion content of the experimental group was significantly increased by about 16% after the CCD-966Sk cells in the experimental group were treated with the Lactobacillus sakei TCI147 sample.

It could be seen from the experimental results that the Lactobacillus sakei TCI147 sample could significantly increase the collagen secretion content, and the ability of the Lactobacillus sakei TCI147 sample to increase the collagen secretion content was significantly superior to that of the MRSD medium. Collagen is found in connective tissue and is a main constituent of the extracellular matrix and cornea. The collagen can provide skin elasticity. In other words, the Lactobacillus sakei TCI147 and/or the metabolites thereof in any embodiment could increase collagen secretion content and skin collagen content, thereby restoring skin rejuvenation and elasticity and preventing wrinkles.

### Example 9

A. Materials and Instruments:

1. Cell line: Human skin fibroblasts, purchased from ATCC, with a cell number of CRL-1881, hereinafter referred to as CCD-966Sk cells.

2. Cell Medium: Minimum essential medium (MEM, purchased from Gibco, with a product number of 11095080), in which 10% (v/v) FBS (purchased from Gibco, with a product number of 10437-028), 1% (v/v) antibiotic (purchased from Gibco, with a product number of 15140122) and 1 mM sodium acetonate (purchased from Gibco, with a product number of 11360-070) were added.

3. Primary Antibody Solution: Collagen type I antibody (purchased from Abcam, with a product number of ab138492) was diluted with a 1% BSA solution in a dilution ratio of 1:250.

4. Secondary Antibody Solution: Anti-mouse-alexa 488 antibody (purchased from Thermo, with a product number of A51011) was diluted with a 1% BSA solution in a dilution ratio of 1:500.

5. Hoechst, purchased from Thermo, with a product number of 62249.

6. 0.2% Triton X-100 Solution: Prepared from Triton X-100 (purchased from Sigma, with a product number of 93443) and PBS.

7. 1% BSA solution: Prepared from BSA (purchased from Sigma, with a product number of A8531) and PBS.

8. Mountant, purchased from Thermo, with a product number of S36936.

9. 4% paraformaldehyde, purchased from Cepham Life Sciences, with a product number of 66311.

10. Fluorescent microscope, purchased from ZEISS, with a model of Vert.A1.

B. Trial Procedures:

1. The CCD-966Sk cells were inoculated into a chamber slide at an amount of 2×10³ cells per well, and were cultured at 37° C. for 24 h. Here, the cells were divided into three trial groups: a blank group, a control group and an experimental group, respectively. Each group underwent trial in triplicate (that is, each group had three wells).

2. After the 24 h of culture, a 0.5% (v/v) MRSD medium was added to the control group, and the 0.5% (v/v) Lactobacillus sakei TCI147 sample prepared in Example 2 was added to the experimental group, and each group was cultured at 37° C. for 24 h.

3. After the 24 h of culture, a supernatant of each group was removed, 4% paraformaldehyde was added to each group, and the cells were fixed at room temperature for 10 min. After the fixation, washing was performed for 3 times with PBS.

4. 0.2% Triton X-100 solution was added to each group, and was left for a reaction at room temperature for 10 min. After the reaction, a supernatant of each group was removed.

5. The 1% BSA solution was added to each group, and was left for a reaction at 37° C. for 1 h. After the reaction, washing was performed for 3 times with PBS.

6. The primary antibody solution was added to each group, and was left for a primary antibody reaction at 37° C. for 1 h. After the primary antibody reaction, washing was performed for 3 times with PBS.

7. The secondary antibody solution was added to each group, and was left for a secondary antibody reaction at 37° C. for 1 h. After the secondary antibody reaction, washing was performed for 3 times with PBS.

8. Hoechst was added to each group, and was left for a reaction at room temperature for 3 to 5 min. After the reaction, washing was performed for 3 times with PBS.

9. Each group was covered with cover glass and mounted with the mountant, and each group was observed with the fluorescent microscope in dark and staining results of the cells in each group were photographed. In the staining results, green fluorescence represented the signal of the collagen, while blue fluorescence represented signals of nuclei.

C. Trial Results:

Refer to FIG. 12. The green fluorescent signals of the blank group were fewer, which indicated that the collagen content in the blank group was lower, which was the expression of the cells under normal physiological metabolism. Relative to those in the blank group, the cells in the control group had more green fluorescent signals, and the brightness of the green fluorescent signals was also slightly improved, which indicated that the collagen content in the control group was slightly higher than that in the blank group. Relative to those in the blank group, the experimental group had more green fluorescent signals, and the brightness of the green fluorescent signals was also higher, which indicated that the collagen content in the experimental group was higher, which was far higher than that in the blank group. Relative to the control group, the experimental group had more green fluorescent signals, and the brightness of the green fluorescent signals was also higher, which indicated that the collagen content in the experimental group was higher, which was far higher than that in the control group.

It could be seen from the experimental results that the Lactobacillus sakei TCI147 sample could significantly increase the collagen content, and the ability of the Lactobacillus sakei TCI147 sample to increase the collagen content was significantly superior to that of the MRSD medium. Collagen is found in connective tissue and is a main constituent of the extracellular matrix and cornea. The collagen can provide skin elasticity. In other words, the Lactobacillus sakei TCI147 and/or the metabolites thereof in any embodiment could increase collagen content, thereby restoring skin rejuvenation and elasticity and preventing wrinkles

### Example 10

A. Trial Procedures:

10 adult subjects to resist aging and improve their skin conditions were asked to continuously take one TCI147 capsule (i.e., a capsule containing 100 mg of Lactobacillus sakei TCI147 live bacteria) before breakfast every day for 8 weeks (i.e., 56 d).

Skin detection and blood drawing were performed before taking (face cleaned, week 0), after taking for 28 d (face cleaned, week 4), and after taking for 56 d (face cleaned, week 8).

The blood drawing was to detect changes in malondialdehyde (MDA) and glutathione S-transferase in red blood cells (GST-RBC) content in blood before and after taking the capsule containing the Lactobacillus sakei TCI147 sample. The MDA and GST-RBC content in the blood of the subject in this embodiment was performed with reference to the blood test standards announced by the Ministry of Health and Welfare (Taiwan).

The skin detection was based on different detection items, values of facial skin were recorded by using corresponding instruments and measurement methods, and the facial skin before and after taking was photographed. Moreover, when detection was performed before and after taking, the temperature and humidity of a detection region where the subject was located were consistent, so as to reduce the impact of external factors such as temperature and humidity on the skin.

Here, skin detection items included skin brown spot, skin fine lines, skin texture, skin collagen density and skin hydration.

The facial skin of the subject before taking, after taking for 28 d and after taking for 56 d was detected for the skin brown spots by using a VISTA Complex Analysis System purchased from the Canfield Scientific in USA. This VISTA Complex Analysis System was used to take images of the facial skin through an RBX light polarization technology, and detect melanotic spots on the dermis invisible to the naked eye to obtain a value that may represent the brown spot condition of the skin (hereinafter referred to as a skin brown spot level value). Moreover, the higher the obtained skin brown spot level value, the higher the skin brown spot level. Then, relative skin brown spot level was calculated with the following formula: relative skin brown spot level (%)=(skin brown spot level value of each group/skin brown spot level value before taking)×100%.

The facial skin of the subject before taking, after taking for 28 d and after taking for 56 d was detected for the skin fine lines by using the VISTA Complex Analysis System purchased from the Canfield Scientific in USA. This VISTA Complex Analysis System was used to take images of the facial skin through a high-resolution camera lens, and could detect the length and depth of the fine lines by irradiating the skin with standard white light and detecting changes in skin shadows, and performed analytical calculation to obtain a value that may represent the fine line condition of the skin (hereinafter referred to as a skin fine line level value). Here, the higher the obtained skin fine line level value, the higher the skin fine line level. Then, relative skin fine line level was calculated with the following formula: relative skin fine line level (%)=(skin fine line level value of each group/skin fine line level value before taking)×100%.

The facial skin of the subject before taking, after taking for 28 d and after taking for 56 d was detected for the skin texture by using the VISIA Complex Analysis System purchased from the Canfield Scientific in USA. This VISIA Complex Analysis System was used to take images of the facial skin through a high-resolution camera lens, and perform analytical calculation on pits and bumps of the detected skin to obtain a value that may represent the roughness condition of the skin (hereinafter referred to as a skin texture level value). Here, the higher the obtained skin texture level value, the higher the skin roughness level. Then, relative skin texture level was calculated with the following formula: relative skin texture level (%)=(skin texture level value of each group/skin texture level value before taking)×100%.

The facial skin of the subject before taking, after taking for 28 d and after taking for 56 d was detected for the skin collagen density by using a High Freq. Ultrasound Module (DermaLab® USB skin analyzer, Denmark) purchased from the Cortex Technology in Denmark. This High Freq. Ultrasound Module was used to transmit acoustic pulse waves into the skin, convert reflected signals of different intensities into different color scales (the lighter or brighter the color, the higher the skin collagen content), and calculate these color scales to obtain a value that may represent the skin collagen density (hereinafter referred to as a skin collagen density value). Here, the higher the obtained skin collagen density value, the higher the skin collagen density. Then, relative skin collagen density was calculated with the following formula: relative skin collagen density (%)=(skin collagen density value of each group/skin collagen density value before taking)×100%.

The facial skin of the subject before taking, after taking for 28 d and after taking for 56 d was detected for the skin hydration by using a skin hydration detection probe Corneometer® CM825 (C+K Multi Probe Adapter System, Germany) purchased from the Courage+Khazaka electronic in Germany. The skin hydration detection probe performed measurement on the principle of capacitance. When the hydration changed, a capacitance value of the skin also changed, and therefore, a value (hereinafter referred to as skin hydration value) that may represent the skin hydration can be obtained by measuring the capacitance value of the skin. Here, the higher the obtained skin hydration value, the higher the skin moisture content. Then, relative skin hydration was calculated with the following formula: relative skin hydration (%)=(skin hydration value of each group/skin hydration value before taking)×100%.

It should be particularly noted that statistically significant differences between the measurements before and after taking were statistically analyzed by a student t-test. In the figure, ‘*’ represents that the p value is less than 0.05 in comparison with before taking, ‘**’ represents that the p value is less than 0.01 in comparison with before taking, and ‘***’ represents that the p value is less than 0.001 in comparison with before taking. The more ‘*’, comes with more significant statistical difference.

B. Trial Results:

1. Detection Results Regarding ‘MDA Content’ of the Subject

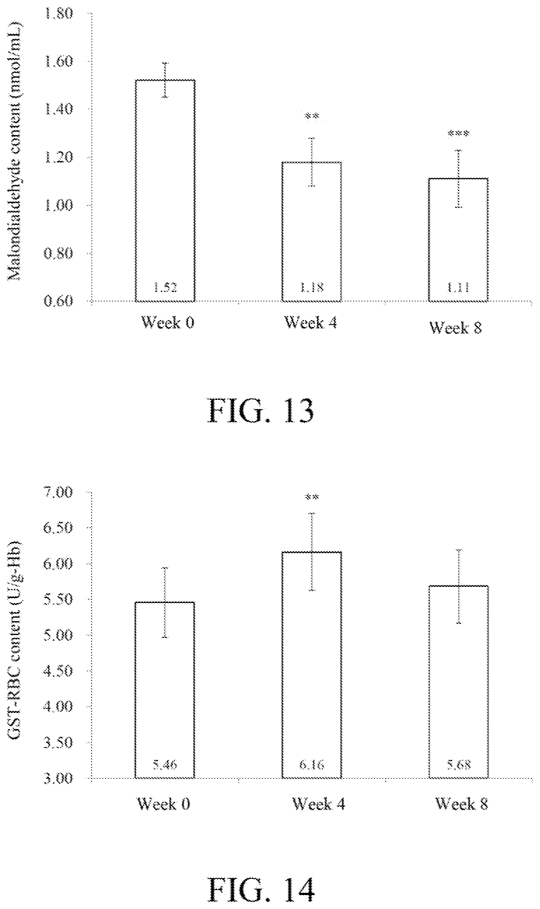

Refer to FIG. 13. The MDA content of the subject at week 0 was about 1.52 nmol/mL, the MDA content of the subject at week 4 decreased to about 1.18 nmol/mL, and the MDA content of the subject at week 8 decreased to about 1.11 nmol/mL. Here, the relative MDA content of the subject at week 0 was set as 100%, and measurements of the subject at weeks 4 and 8 were correspondingly converted into the relative MDA content, which could obtain that the relative MDA content of the subject at week 4 (after continuously taking the Lactobacillus sakei TCI147 sample for 4 weeks) was significantly reduced to about 77.6%, with a decrease of 22.4%, and the relative MDA content of the subject at week 8 (after continuously taking the Lactobacillus sakei TCI147 sample for 8 weeks) was significantly reduced to about 73.0%, with a decrease of 27.0%. Moreover, the proportion of people improved reached 90%. Malondialdehyde is an indicator of oxidative damage in blood. It could be seen herefrom that the Lactobacillus sakei TCI147 and/or the metabolites thereof in any embodiment could reduce malondialdehyde, enhance antioxidant capacity in the body, thereby resisting physiological aging.

2. Detection Results Regarding ‘GST-RBC Content’ of the Subject

Refer to FIG. 14. The GST-RBC content of the subject at week 0 was about 5.46 U/g-Hb, the GST-RBC content of the subject at week 4 increased to about 6.16 U/g-Hb, and the GST-RBC content of the subject at week 8 increased to about 5.68 U/g-Hb. Here, the relative GST-RBC content of the subject at week 0 was set as 100%, and measurements of the subject at weeks 4 and 8 were correspondingly converted into the relative GST-RBC content, which could obtain that the relative GST-RBC content of the subject at week 4 (after continuously taking the Lactobacillus sakei TCI147 sample for 4 weeks) was significantly increased to about 112.8%, with an increase of 12.8%, and the relative GST-RBC content of the subject at week 8 (after continuously taking the Lactobacillus sakei TCI147 sample for 8 weeks) was significantly increased to about 104.0%, with an increase of 4.0%. Moreover, the proportion of people improved reached 70%. Glutathione S-transferase is an antioxidant substance. It could be seen herefrom that the Lactobacillus sakei TCI147 and/or the metabolites thereof in any embodiment could increase glutathione S-transferase, thereby improving antioxidant capacity and immunity in the body, which reduced hydrogen peroxide in the body to water and oxygen, and reduced lipid peroxides to harmless products, so that oxidative stress in the body were reduced to resist physiological aging.

3. Detection Results Regarding ‘Skin Brown Spots’ of Subjects

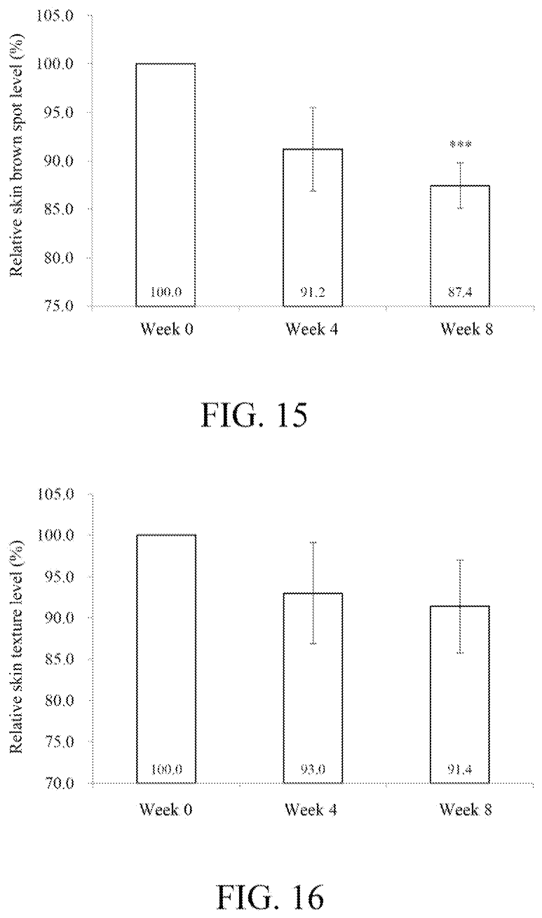

Refer to FIG. 15. The brown spot conditions measured from 10 subjects before taking were regarded as 100% relative skin brown spot level. At this time, an average relative skin brown spot level at week 4 (i.e., after continuously taking the Lactobacillus sakei TCI147 sample for 4 weeks) was 91.2%, and an average relative skin brown spot level at week 8 (i.e., after continuously taking the Lactobacillus sakei TCI147 sample for 8 weeks) was 87.4%. In other words, compared with that before taking, the relative skin brown spot level of these subjects could be reduced by 8.8% after continuous taking of the Lactobacillus sakei TCI147 sample for 4 weeks, while the relative skin brown spot level of these subjects could be significantly reduced by 12.6% after continuous taking of the Lactobacillus sakei TCI147 sample for 8 weeks. Moreover, the proportion of people improved reached 100%. Spots are one of the main symptoms of skin aging. It could be seen herefrom that the Lactobacillus sakei TCI147 and/or the metabolites thereof in any embodiment could indeed significantly reduce and/or fade skin spots, reduce and/or fade skin brown spots or deep-layer spots, improve skin conditions of the subjects, and improve and/or delay skin aging, thereby achieving a skin-care effect.

4. Detection Results Regarding ‘Skin Texture’ of Subjects

Refer to FIG. 16. The skin texture conditions measured from 10 subjects before taking were regarded as 100% relative skin texture level. At this time, an average relative skin texture level at week 4 (i.e., after continuously taking the Lactobacillus sakei TCI147 sample for 4 weeks) was 93.0%, and an average relative skin texture level at week 8 (i.e., after continuously taking the Lactobacillus sakei TCI147 sample for 8 weeks) was 91.4%. In other words, compared with that before taking, the relative skin texture level of these subjects could be reduced by 7.0% after continuous taking of the Lactobacillus sakei TCI147 sample for 4 weeks, while the relative skin texture level of these subjects could be reduced by 8.6% after continuous taking of the Lactobacillus sakei TCI147 sample for 8 weeks. Moreover, the proportion of people improved reached 70%. It could be seen herefrom that the Lactobacillus sakei TCI147 and/or the metabolites thereof in any embodiment could indeed improve skin texture, delay and/or reduce skin roughness, to make the skin smooth and glossy, and improve skin conditions of the subjects, thereby achieving a skin-care effect.

5. Detection Results Regarding ‘Skin Fine Lines’ of Subjects

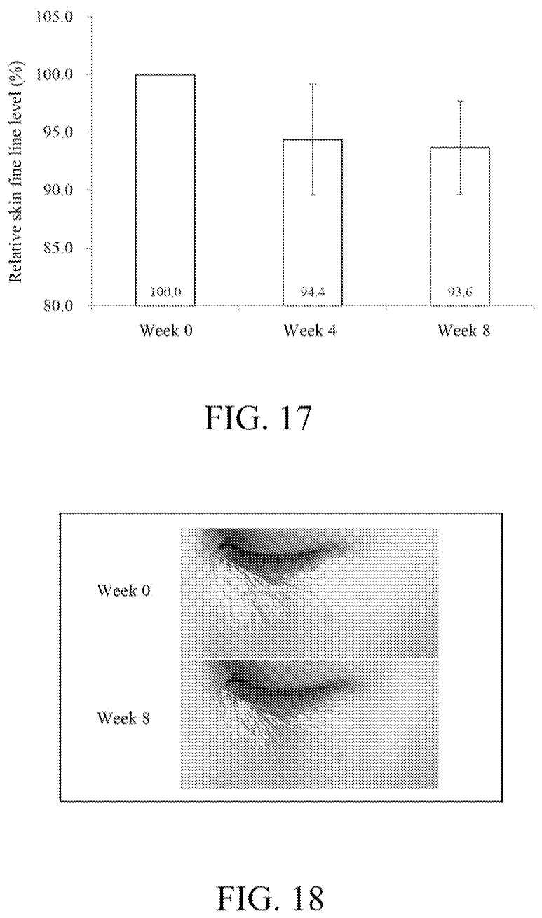

Refer to FIG. 17. The skin fine line conditions measured from 10 subjects before taking were regarded as 100% relative skin fine line level. At this time, an average relative skin fine line level at week 4 (i.e., after continuously taking the Lactobacillus sakei TCI147 sample for 4 weeks) was 94.4%, and an average relative skin fine line level at week 8 (i.e., after continuously taking the Lactobacillus sakei TCI147 sample for 8 weeks) was 93.6%. In other words, compared with that before taking, the relative skin fine line level of these subjects could be reduced by 5.6% after continuous taking of the Lactobacillus sakei TCI147 sample for 4 weeks, while the relative skin fine line level of these subjects could be reduced by 6.4% after continuous taking of the Lactobacillus sakei TCI147 sample for 8 weeks. Moreover, the proportion of people improved reached 60%. It could be seen herefrom that the Lactobacillus sakei TCI147 and/or the metabolites thereof in any embodiment could indeed reduce skin wrinkles and skin fine lines, reduce and/or smooth fine lines around the eyes, and improve skin conditions of the subjects, thereby achieving a skin-care effect.

Refer to FIG. 18. FIG. 18 shows photos of fine lines around eyes of one of the subjects measured at week 0 (before taking) and week 8 (i.e., after continuously taking the Lactobacillus sakei TCI147 sample for 8 weeks). At week 0, there were many fine lines around the eyes. Relative to week 0, there were fewer fine lines around the eyes at week 8 (i.e., after continuously taking the Lactobacillus sakei TCI147 for 8 weeks). In other words, compared with those before taking, the fine lines around the eyes of these subjects could be reduced after continuous taking of the Lactobacillus sakei TCI147 sample for 8 weeks. It could be seen herefrom that the Lactobacillus sakei TCI147 and/or the metabolites thereof in any embodiment could indeed reduce skin wrinkles and skin fine lines, reduce and/or smooth fine lines around the eyes, and improve skin conditions of the subjects, thereby achieving a skin-care effect.

6. Detection Results Regarding ‘Skin Collagen Density’ of Subjects

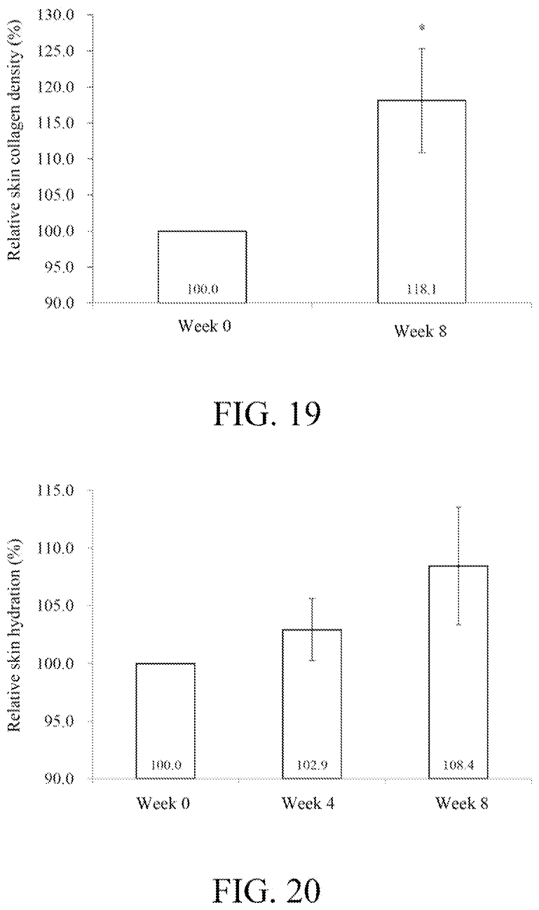

Refer to FIG. 19. The skin collagen density measured from 10 subjects before taking was regarded as 100% relative skin collagen density. At this time, an average relative skin collagen density at week 8 (i.e., after continuously taking the Lactobacillus sakei TCI147 sample for 8 weeks) was 118.1%. In other words, in comparison with that before taking, the relative skin collagen density of these subjects could be significantly increased by 18.1% after continuous taking of the Lactobacillus sakei TCI147 sample for 8 weeks. Moreover, the proportion of people improved reached 90%. It could be seen herefrom that the Lactobacillus sakei TCI147 and/or the metabolites thereof in any embodiment could indeed increase collagen, increase skin collagen content, effectively promote proliferation of collagen, thereby providing skin elasticity and flexibility, making the skin elastic, and improve skin conditions of the subjects, thereby achieving a skin-care effect.

7. Detection Results Regarding ‘Skin Hydration’ of Subjects

Refer to FIG. 20. The skin hydration measured from 10 subjects before taking was regarded as 100% relative skin hydration. At this time, average relative skin hydration at week 4 (i.e., after continuously taking the Lactobacillus sakei TCI147 sample for 4 weeks) was 102.9%, and average relative skin hydration at week 8 (i.e., after continuously taking the Lactobacillus sakei TCI147 sample for 8 weeks) was 108.4%. In other words, compared with that before taking, the relative skin hydration of these subjects could be reduced by 2.9% after continuous taking of the Lactobacillus sakei TCI147 sample for 4 weeks, while the relative skin hydration of these subjects could be reduced by 8.4% after continuous taking of the Lactobacillus sakei TCI147 sample for 8 weeks. Moreover, the proportion of people improved reached 60%. It could be seen herefrom that the Lactobacillus sakei TCI147 and/or the metabolites thereof in any embodiment could indeed increase skin hydration, promote skin moisture retention capacity, and improve skin conditions of the subjects, thereby achieving a skin-care effect.

### Example 11

A. Materials and Instruments:

1. Strains in Table III, a total of 14 strains, including 4 Lactobacillus sakei strains, 5 Bacillus coagulans strains and 5 Lactobacillus casei strains.

TABLE III

| Strain | | 16S rDNA | |
|---|---|---|---|
| Number | Strain | Sequence Number | Isolated from |
| TCI147 | Lactobacillus sakei | SEQ ID NO: 1 | Lentinus edodes |
| LP141 | Lactobacillus sakei | SEQ ID NO: 12 | Flammulina velutipes |
| LP144 | Lactobacillus sakei | SEQ ID NO: 13 | Cordyceps sobolifera |
| | | | fruiting bodies |
| LP156 | Lactobacillus sakei | SEQ ID NO: 14 | Canarium pimela |
| LF018 | Bacillus coagulans | SEQ ID NO: 15 | Ginger |
| LF358 | Bacillus coagulans | SEQ ID NO: 16 | Breast milk |
| LP236 | Bacillus coagulans | SEQ ID NO: 17 | Infant feces |
| LP223 | Bacillus coagulans | SEQ ID NO: 18 | Hydrangea |
| | | | macrophylla |
| L0001 | Bacillus coagulans | SEQ ID NO: 19 | Amaranthus tricolor |
| LF320 | Lactobacillus casei | SEQ ID NO: 20 | Prosciutto crudo |
| LF410 | Lactobacillus casei | SEQ ID NO: 21 | Muenster Cheese |
| LF348 | Lactobacillus casei | SEQ ID NO: 22 | Ananas comosus |
| LF414 | Lactobacillus casei | SEQ ID NO: 23 | Pickles |
| LP403 | Lactobacillus casei | SEQ ID NO: 24 | Prosciutto crudo |

2. MRSD medium, purchased from BD, with a product number of 288130.

3. Spermidine ELISA Kit, purchased from Cloud-Clone, with a product number of CEX053Ge.

B. Trial Procedures:

1. The strains in Table III were inoculated into the MRSD medium with 1% (v/v) of bacterial load (about 1×10⁴ CFU/mL), and were cultured at 37° C. for 24 h to form a bacterial solution of each strain.

2. The bacterial solution of each strain was centrifuged at 5,000 rpm for 10 min to obtain a supernatant of each strain, and the spermidine production of each strain was determined by using the spermidine ELISA kit. Here, after the supernatant of each strain was treated according to the trial procedures provided by the spermidine ELISA kit, an absorbance value at 450 nm (OD₄₅₀ value) per well was measured by using the ELISA reader.

C. Trial Results:

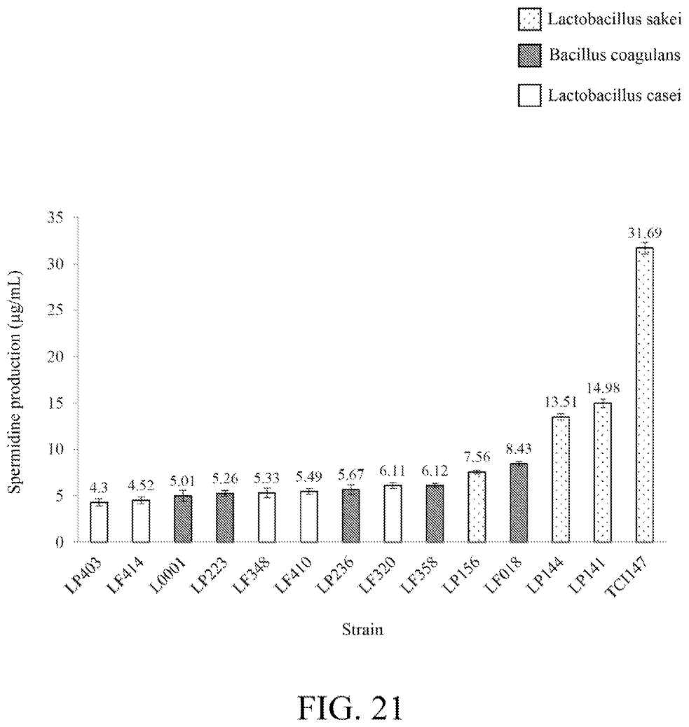

Refer to FIG. 21. The spermidine production of LP403 was 4.3 ug/mL (ppm); the spermidine production of LF414 was 4.52 ug/mL (ppm); the spermidine production of L0001 was 5.01 ug/mL (ppm); the spermidine production of LP223 was 5.26 ug/mL (ppm); the spermidine production of LF348 was 5.33 ug/mL (ppm); the spermidine production of LF410 was 5.49 ug/mL (ppm); the spermidine production of LP236 was 5.67 ug/mL (ppm); the spermidine production of LF320 was 6.11 ug/mL (ppm); the spermidine production of LF358 was 6.12 ug/mL (ppm); the spermidine production of LP156 was 7.56 ug/mL (ppm); the spermidine production of LF018 was 8.43 ug/mL (ppm); the spermidine production of LP144 was 13.51 ug/mL (ppm); the spermidine production of LP141 was 14.98 ug/mL (ppm); and the spermidine production of TCI147 was 31.69 ug/mL (ppm). That is, relative to that of other Lactobacillus casei, Bacillus coagulans and Lactobacillus sakei strains, the spermidine production of the Lactobacillus sakei TCI147 was significantly increased by about 111.55% to 636.98%. Relative to that of the Lactobacillus casei, the spermidine production of the Lactobacillus sakei TCI147 was significantly increased by about 418.66% to 636.98%. Relative to that of the Bacillus coagulans, the spermidine production of the Lactobacillus sakei TCI147 was significantly increased by about 275.92% to 532.53%. Relative to that of other Lactobacillus sakei strains, the spermidine production of the Lactobacillus sakei TCI147 was significantly increased by about 111.55% to 319.18%.

It could be seen from the experimental results that the Lactobacillus sakei TCI147 could significantly increase the spermidine production, and the ability of the Lactobacillus sakei TCI147 to increase the spermidine production was significantly superior to that of other Lactobacillus casei, Bacillus coagulans and Lactobacillus sakei strains. Spermidine is a polyamine crystalline compound, which can effectively induce the autophagy, improve the mitochondrial function and activity, and has been proved to be a potential anti-aging substance. Literature pointed out that spermidine has various anti-aging effects, including memory loss prevention, mitochondrial activity acceleration, skin barrier repair, retinal cell repair, cardiovascular health care and the like. In other words, the Lactobacillus sakei TCI147 and/or the metabolites thereof in any embodiment could increase spermidine production, increase spermidine, thereby effectively inducing autophagy, improving mitochondrial function and activity, preventing memory loss, repairing skin barrier, repairing retinal cells, realizing cardiovascular health care and the like. The Lactobacillus sakei TCI147 and/or the metabolites thereof in any embodiment has an anti-aging capacity.

In summary, the Lactobacillus sakei in any embodiment can produce spermidine. In some embodiments, the Lactobacillus sakei or metabolites thereof in any embodiment is suitable for preparing a composition for spermidine production. In some embodiments, a method for spermidine production includes administering to a subject in need thereof a composition including the Lactobacillus sakei or the metabolites thereof in any embodiment. In other words, the aforementioned composition has a spermidine production function. In some embodiments, the Lactobacillus sakei, the metabolites thereof or the composition prepared therefrom further has one or more of the following functions: delaying cellular aging or oxidation, enhancing mitochondrial activity, promoting autophagy, preventing and/or reducing oxidative stress of the cells, increasing GSH content, enhancing GST activity, reducing MDA content, increasing GST-RBC content, delaying skin aging, increasing skin collagen content, fading skin spots, reducing skin roughness, reducing skin fine lines and/or wrinkles and promoting skin moisture retention capacity. In some embodiments, a method for delaying cellular aging or oxidation, enhancing mitochondrial activity, promoting autophagy, preventing and/or reducing oxidative stress of the cells, increasing GSH content, enhancing GST activity, reducing MDA content, increasing GST-RBC content, delaying skin aging, increasing skin collagen content, fading skin spots, reducing skin roughness, reducing skin fine lines and/or wrinkles and promoting skin moisture retention capacity includes administering to a subject in need thereof a composition including the Lactobacillus sakei or the metabolites thereof

What is claimed is:

1. Lactobacillus sakei, wherein the Lactobacillus sakei is Lactobacillus sakei TCI147 with an accession number of DSM 33913.

2. A method for spermidine production, comprising administrating to a subject in need thereof a composition comprising Lactobacillus sakei or a metabolite thereof, wherein the Lactobacillus sakei is Lactobacillus sakei TCI147 with an accession number of DSM 33913.

3. The method according to claim 2, wherein the Lactobacillus sakei is used for delaying cellular aging or oxidation.

4. The method according to claim 2, wherein the Lactobacillus sakei is used for enhancing mitochondrial activity.

5. The method according to claim 2, wherein the Lactobacillus sakei is used for promoting autophagy.

6. The method according to claim 3, wherein the Lactobacillus sakei is used for preventing and/or reducing oxidative stress of cells.

7. The method according to claim 3, wherein the Lactobacillus sakei is used for increasing glutathione (GSH) content.

8. The method according to claim 3, wherein the Lactobacillus sakei is used for enhancing glutathione S-transferase (GST) activity.

9. The method according to claim 3, wherein the Lactobacillus sakei is used for reducing malondialdehyde (MDA) content and/or increasing glutathione S-transferase in red blood cells (GST-RBC) content.

10. The method according to claim 3, wherein the Lactobacillus sakei is used for delaying skin aging.

11. The method according to claim 2, wherein the Lactobacillus sakei is used for increasing skin collagen content.

12. The method according to claim 2, wherein the Lactobacillus sakei is used for fading skin spots.

13. The method according to claim 2, wherein the Lactobacillus sakei is used for reducing skin roughness.

14. The method according to claim 2, wherein the Lactobacillus sakei is used for reducing skin fine lines and/or wrinkles.

15. The method according to claim 2, wherein the Lactobacillus sakei is used for promoting moisture retention capacity.
